(19) United States
(12) Patent Application Publication
(10) Pub. No.: US 2024/0191276 A1
(43) Pub. Date: Jun. 13, 2024

(54) METHODS FOR LIGATION OF (POLY)PEPTIDES AND OLIGONUCLEOTIDES

(71) Applicant: Nanyang Technological University, Singapore (SG)

(72) Inventors: Anh Tuan PHAN, Singapore (SG); Derrick Jing Yang TAN, Singapore (SG); Kah Wai LIM, Singapore (SG); Vee Vee CHEONG, Singapore (SG)

(21) Appl. No.: 18/547,468

(22) PCT Filed: Feb. 23, 2022
(86) PCT No.: PCT/SG2022/050088

§ 371 Date: Aug. 22, 2023

(30) Foreign Application Priority Data
Feb. 23, 2021 (SG) 10202101791Y

(51) Int. Cl. C12P 21/02; C12N 9/00; C12N 9/52

(52) U.S. Cl. CPC C12P 21/02; C12N 9/52; C12N 9/93; C12Y 304/2207; C12Y 601/01022

(57) ABSTRACT

The present invention lies in the technical field of enzymatic (poly)peptide ligation and specifically relates to methods that allow the ligation of (poly)peptides and oligonucleotides. The methods comprise providing at least one cargo molecule modified with a peptide tag and at least one poly(peptide) to be ligated to the cargo molecule, wherein the peptide tag and/or the (poly)peptide comprises a ligation motif for a peptide ligase, preferably sortase and peptidyl asparaginyl ligases (PALs), such as butelase-1, VyPAL2 or OaAEPI b. The invention also relates to the resulting conjugates and the corresponding uses.

### CROSS-REFERENCE TO RELATED APPLICATIONS

This application claims the benefit of priority of Singapore Patent Application No. 10202101791Y filed Feb. 23, 2021, the content of which being hereby incorporated by reference in its entirety for all purposes.

### Field of the Invention

The present invention lies in the technical field of enzymatic (poly)peptide ligation and specifically relates to methods that allow the ligation of (poly)peptides and oligonucleotides. The invention also relates to the resulting conjugates and the corresponding uses.

### BACKGROUND OF THE INVENTION

Peptides and oligonucleotides (oligos) are widely utilized as chemical and molecular biology tools, and they have emerged as viable and effective drug classes in recent years. Joining the two modalities would provide an opportunity to harness and combine the benefits of both, and to expand the scope of their activity. For instance, peptides can carry oligos, and conversely, aptamers can carry peptides, over to their desired sites of action in vivo or in cells to serve as therapeutics or Imaging tools. However, due to limited compatibility in chemistry between the solid-phase synthesis of peptides and oligos generation of peptide-oligo conjugates (POCs) remains an ad hoc and laborious process. A streamlined method towards the ligation of peptides and oligos would thus offer a means to expand the chemical biology toolbox and facilitate the production of POCs for therapeutic development.

To date, two general strategies are available for the synthesis of POCs (Tung & Stein Bioconjug Chem 2000, 11 (5), 605-18; MacCulloch et al. Org Biomol Chem 2019, 17 (7), 1668-1682; Venkatesan & Kim Chem. Rev. 2006, 106 (9), 3712-61; Lu et al. Bioconjug Chem 2010, 21 (2), 187-202). The first involves in-line synthesis through sequential addition of amino acid and nucleotide monomers onto the same solid support (Haralambidis et al. Tetrahedron Lett. 1987, 28, 5199-5202; Bergmann et al. Tetrahedron Lett. 1995, 36, 1839-1842; Soukchareun et al. Bioconjug Chem 1995, 6 (1), 43-53; Truffert et al. Tetrahedron Lett. 1994, 35, 2353-2356; de la Torre et al. Tetrahedron Lett. 1994, 35, 2733-2736; Zaramella et al. J. Am. Chem. Soc. 2004, 126 (43), 14029-35; Ocampo et al. Org Lett 2005, 7 (20), 4349-52; Stetsenko et al. Org Lett 2002, 4 (19), 3259-62; Antopolsky et al. Tetrahedron Lett 2002, 43 (3), 527-530). Albeit straightforward, this strategy is constrained by limited choices of compatible protecting groups, given that contrasting chemistries are employed in the stepwise coupling and global deprotection of the two entities. On the other hand, post-synthetic conjugation involves synthesis, deprotection, and purification of peptide and oligonucleotide separately before their conjugation (Eritja et al. Tetrahedron 1991, 47, 4113-4120; Corey Methods Mot Biol 2004, 283, 197-206; Astakhova et al. Org Biomol Chem 2013, 11 (25), 4240-9; Sanchez et al. Bioconjugate Chem. 2012, 23 (2), 300-7; Taskova et al. Bioconjug Chem 2017, 28 (3), 768-774; Bongartz et al. Nucleic Acids Res. 1994, 22(22), 4681-8). While this circumvents the compatibility issue, multiple synthesis and purification steps often render this approach tedious and yield-limiting. Moreover, the conjugation chemistry chosen must be exclusively orthogonal to prevent side reactions with reactive side chains of the peptide.

Peptide ligases catalyze the joining of two peptide ends that contain matching sequence or chemical motifs. They have been extensively applied in protein engineering (Schmidt et al. Curr Opin Chem Biol 2017, 38, 1-7), ranging from macrocyclization (Harris et al. Sci. Rep. 2019, 9 (1), 10820; Nguyen et al. Nat. Chem. Biol. 2014, 10 (9), 732-8; Wu et al. Chem Commun (Camb) 2011, 47 (32), 9218-20; Schmidt et al. ChemBioChem 2019, 20 (12), 1524-1529) and site-specific labeling of peptides (Schumacher et al. Angew. Chem. Int. Ed. Engl. 2015, 54 (46), 13787-91; Rehm et al. J. Am. Chem. Soc. 2019, 141 (43), 17388-17393; Antos et al. J. Am. Chem. Soc. 2009, 131 (31), 10800-10801; Weeks & Wells Nat Chem Biol 2018, 14 (1), 50-57; Chen et al. Sci. Rep. 2016, 6, 31899) to intermolecular peptide and protein ligation (Nguyen et al. Angew. Chem. Int. Ed. Engl. 2015, 54 (52), 15694-8; Tan et al.Org Lett2018, 20 (21), 6691-6694; Henager Nat Methods 2016, 13 (11), 925-927).

The use of an enzyme, which exhibits a high regioselectivity, ensures a clean reaction while also allowing the ligation to proceed under mild aqueous conditions that are compatible with most folded peptides and proteins. Adaptation of such enzymatic approaches towards post-synthetic generation of POCs would thus be highly desirable. However, few reports of POC creation utilizing this approach are available (Koussa et al. Methods 2014, 67 (2), 134-41; Harmand et al. Bioconjug Chem 2018, 29 (10), 3245-3249) and there remains a need for a streamlined process to the creation of diverse POCs.

The present invention meets that existing need in that it provides a phosphoramidite tag-based approach to enzymatic ligation of peptides and oligonucleotides that greatly simplifies the preparation of POCs.

### SUMMARY OF THE INVENTION

In a first aspect, the present invention is directed to methods for the enzymatic ligation of a (poly)peptide with a cargo molecule, the method comprising the steps of:
(i) providing at least one cargo molecule modified with a peptide tag and at least one poly(peptide) to be ligated to the cargo molecule, wherein the peptide tag and/or the (poly)peptide comprises a ligation motif for a peptide ligase; and
(ii) contacting the cargo molecule and the poly(peptide) with a peptide ligase that ligates the peptide tag with the (poly)peptide via the ligation motif.

In various embodiments, the cargo molecule comprises one or more peptide tags. The peptide tag may be up to 10 amino acids in length, preferably 2 to 7 amino acids in length. The peptide tag may be coupled to the cargo molecule via a scaffold moiety.

In various embodiments, the ligation motif for a peptide ligase is located on the C-terminus of the (poly)peptide to be ligated.

In various embodiments, the peptide ligase is selected from sortase and peptidyl asparaginyl ligases (PALs). Suitable PALs include, but are not limited to butelase-1, VyPAL2 and OaAEP1b.

The cargo molecule may be selected from the group consisting of dyes, drugs, aptamers and oligonucleotides.

In various embodiments, the cargo molecule is an oligonucleotide. In such embodiments, the peptide tag can be attached to the 5′ end, the 3′ end or incorporated into the nucleotide chain of the oligonucleotide or, if multiple peptide tags are used, any combination thereof. The peptide tag may be coupled to the backbone, the sugar or the base moieties of the oligonucleotide or, if multiple peptide tags are used, any combination thereof.

In various embodiments, the oligonucleotide is an oligonucleotide analogue comprising at least one modified base, at least one modified sugar, at least one phosphorothioate linkages, at least one phosphorodiamidate morpholino unit, at least one locked nucleic acid monomer or a combinations thereof.

In various embodiments, the oligonucleotide modified with a peptide tag is obtained by reacting the peptide tag conjugated to a scaffold comprising a reactive group that can be coupled to a nucleotide or nucleotide analogue, preferably a phosphoramidite or phosphoramidate group, with the oligonucleotide under conditions that allow coupling of said reactive group with the oligonucleotide to yield the oligonucleotide modified with a peptide tag. The scaffold may be an amino acid analogue, preferably 4-hydroxyprolinol, serinol, threoninol, N-methyl-serinol, or N-methylthreoninol, each comprising a phosphoramidite or phosphoramidate group.

In various embodiments, where the peptide tag is connected to the scaffold via a carbonyl group, for example its C-terminus, the scaffold may be selected from the group consisting of:

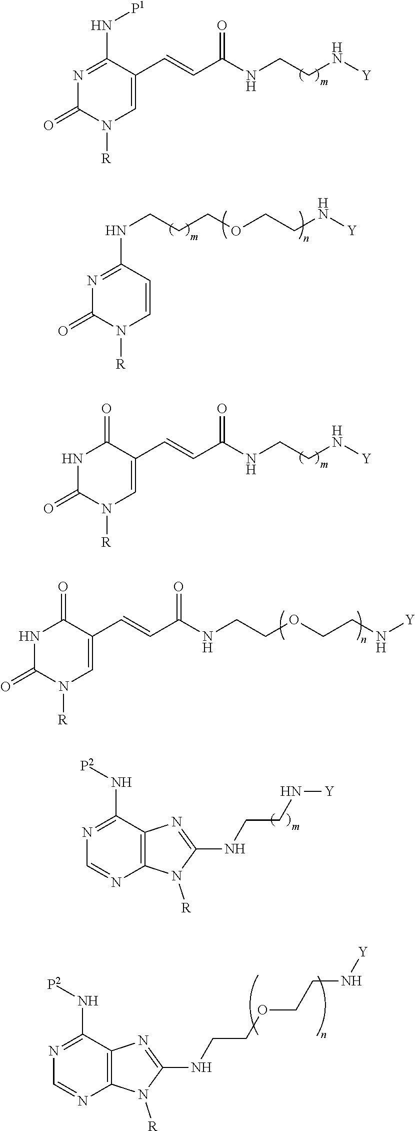

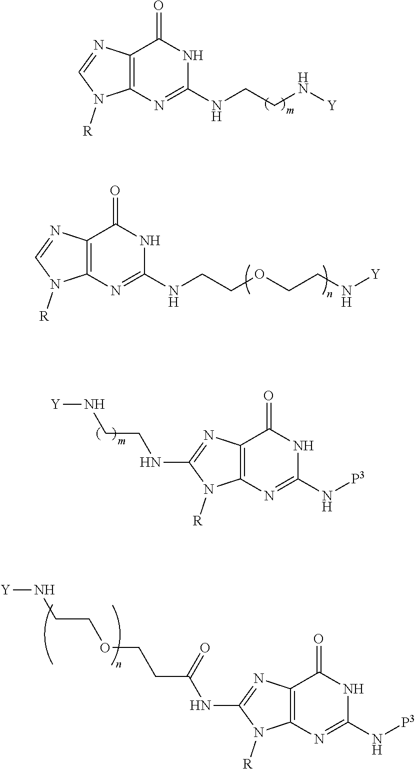

In such embodiments, R may be selected from

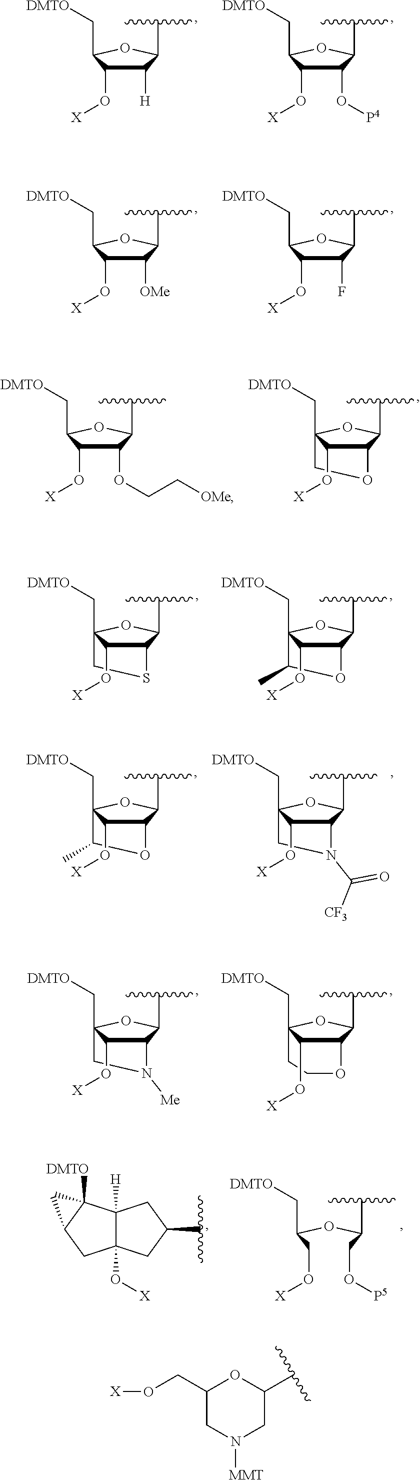

In various other embodiments, where the peptide tag is connected to the scaffold via a carbonyl group, for example its C-terminus, the scaffold may be selected from the group consisting of:

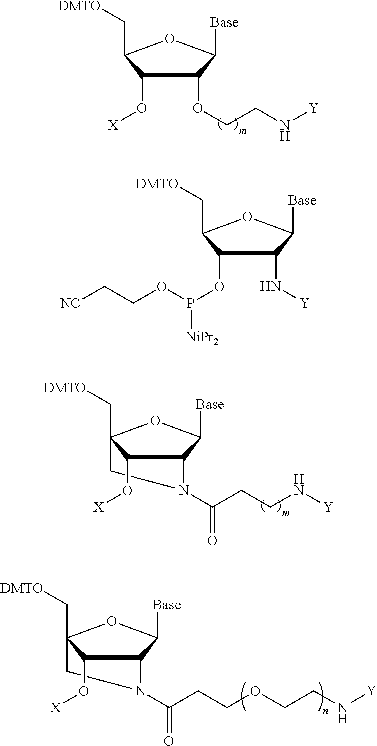

In still other embodiments, where the peptide tag is connected to the scaffold via a carbonyl group, for example its C-terminus, the scaffold may be selected from the group consisting of:

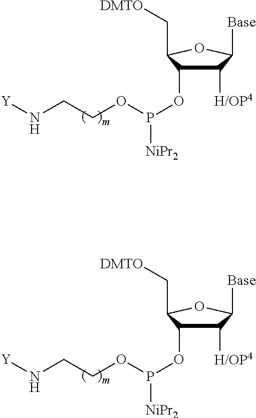

In still other embodiments, where the peptide tag is connected to the scaffold via a carbonyl group, for example its C-terminus, the scaffold is for labeling any position of the oligonucleotide and may be selected from the group consisting of:

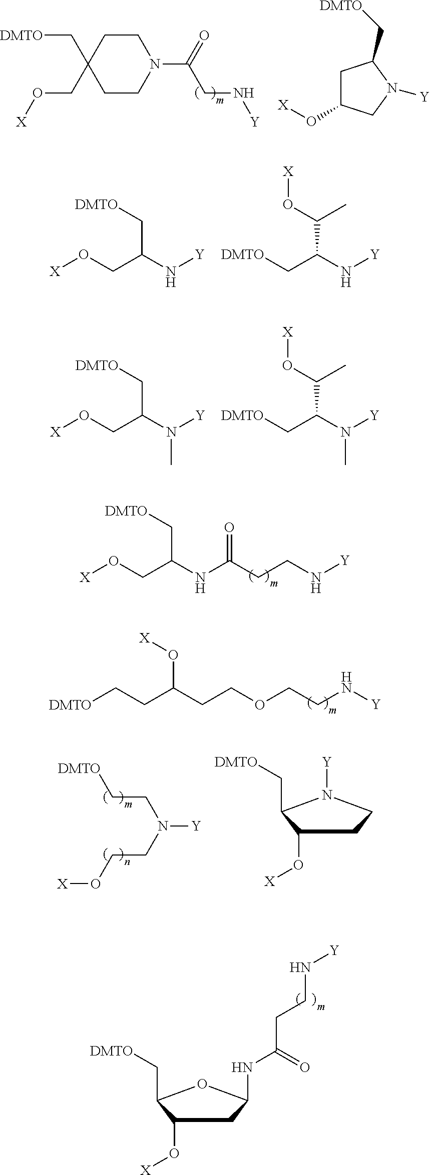

In still other embodiments, where the peptide tag is connected to the scaffold via a carbonyl group, for example its C-terminus, the scaffold is for labeling the terminal position of the oligonucleotide and may be selected from the group consisting of:

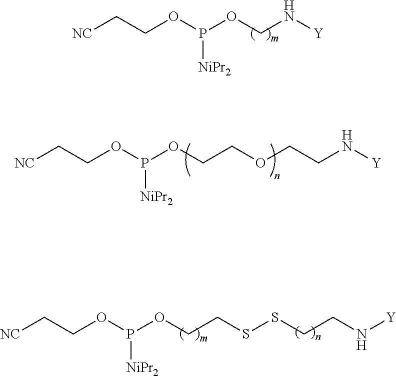

In the above structures, the linkers are shown with DMT (dimethoxytrityl) as a protecting group, which can be interchanged with other suitable protecting groups, including but not limited to MMT (monomethoxytrityl), Trt (trityl), and 2-Cl-Trt (2-chloro-trityl). In these structures, X represents any compatible group that can be effectively coupled onto nucleotide (or nucleotide analogue, e.g. morpholino) chain with solid-phase oligonucleotide synthesis (SPOS) (e.g. a phosphoramidite and phosphoramidate group), or alternatively, the solid support (e.g. solid support-C(═O)—(CH₂)₂—C(═O)—) of SPOS. Y represents any tag motif, including but not limited to those shown below.

In the above structures
m is 0-8
n is 0-8
P¹ is any protecting group on N4 of cytosine (e.g. dmf (N,N-dimethylformamide), Bz (benzyl), Ac (acetyl)) suitable for use in SPOS;
P² is any protecting group on N6 of adenine (e.g. Bz, Pac (phenoxyacetyl)) suitable for use in SPOS;
P³ is any protecting group on N2 of guanine (e.g. iBu (isobutyryl), dmf, iPr-Pac (isopropylphenoxyacetyl)) suitable for use in SPOS;
P⁴ is any protecting group on 2′-O of pentose sugar (e.g. TOM (2-O-triisopropylsilyloxymethyl), TBDMS (2′-O-tert-butyldimethylsilyl), Ac) suitable for use in SPOS of RNA;
P⁵ is any protecting group (e.g. Bz) suitable for use in SPOS of UNA.

In these structures Y is the peptide tag, linked via its C-terminus, and may be selected from the following:

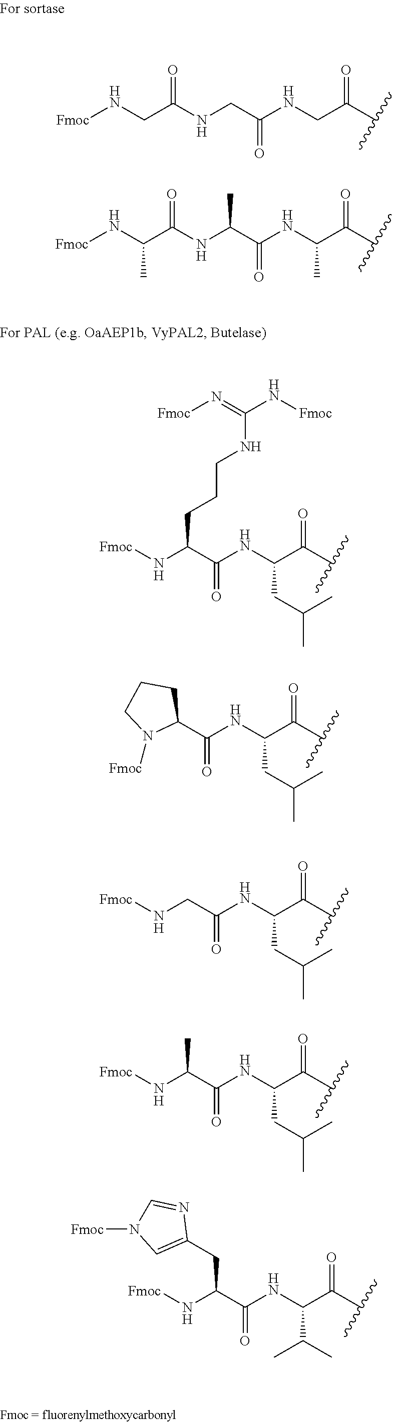

In various embodiments, where the peptide tag is connected to the scaffold via an amino group, for example its N-terminus, the scaffold may be selected from the group consisting of:

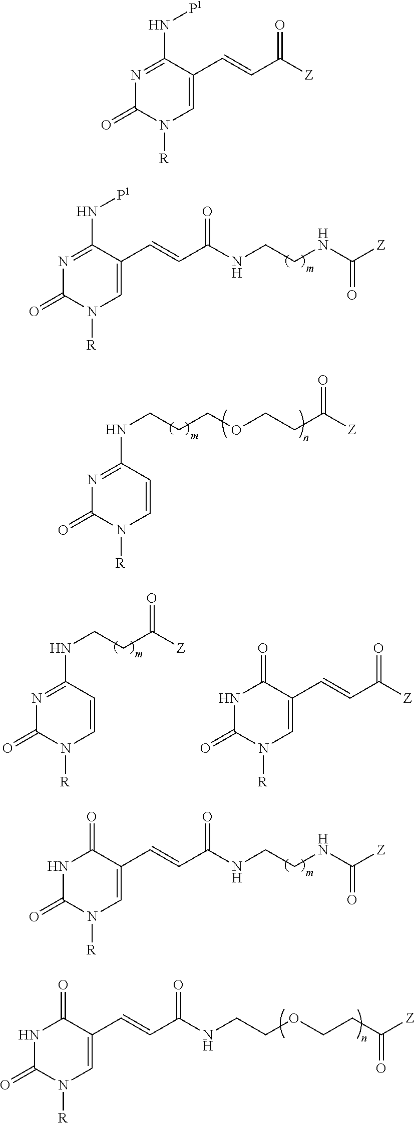

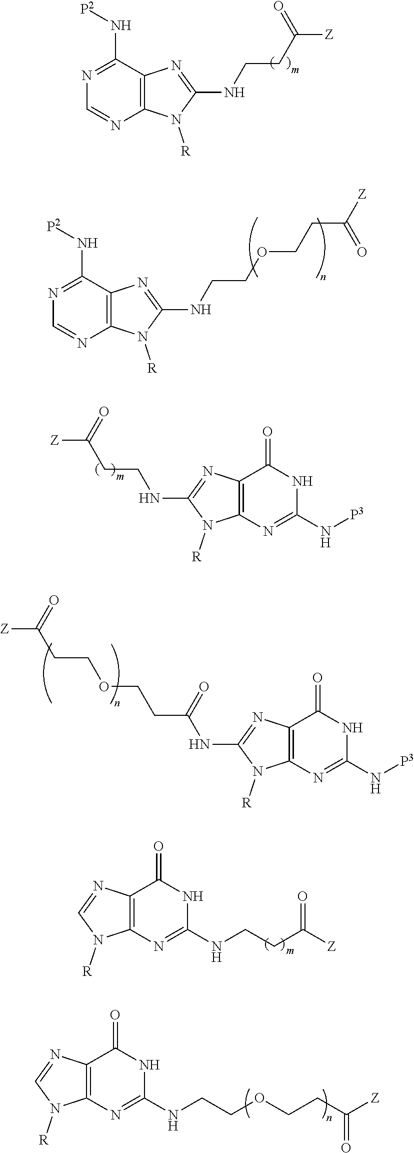

In such embodiments, R may be selected from

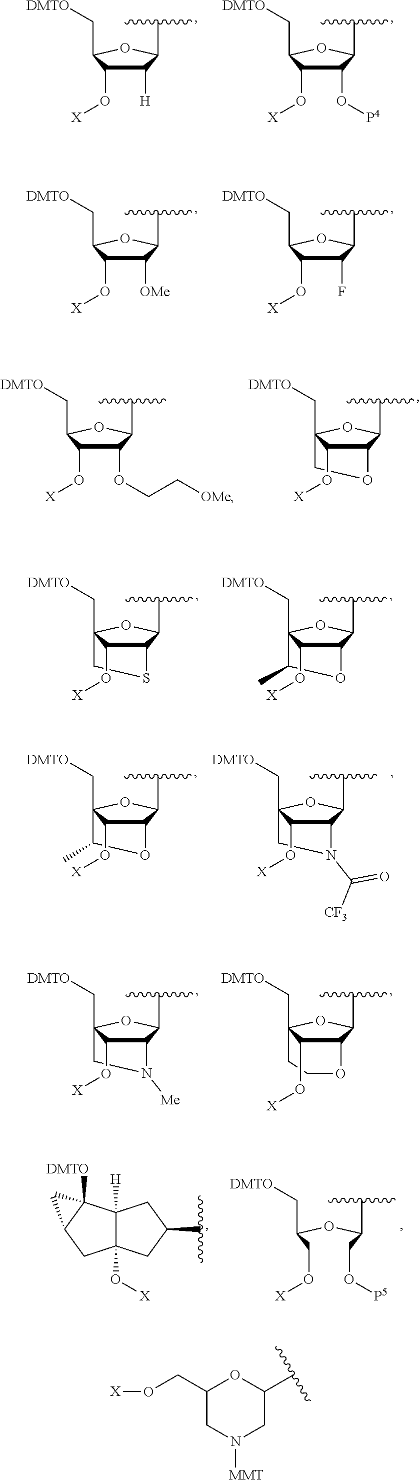

In various other embodiments, where the peptide tag is connected to the scaffold via an amino group, for example its N-terminus, the scaffold may be selected from the group consisting of:

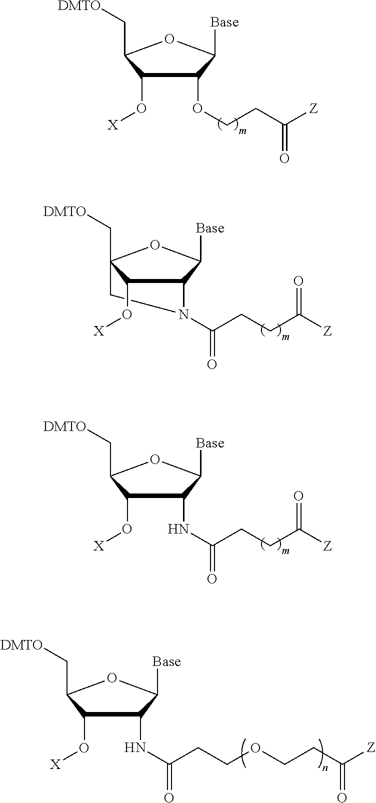

In still other embodiments, where the peptide tag is connected to the scaffold via an amino group, for example its N-terminus, the scaffold may be selected from the group consisting of:

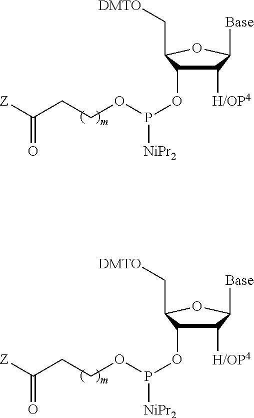

In still other embodiments, where the peptide tag is connected to the scaffold via an amino group, for example its N-terminus, the scaffold is for labeling any position of the oligonucleotide and may be selected from the group consisting of:

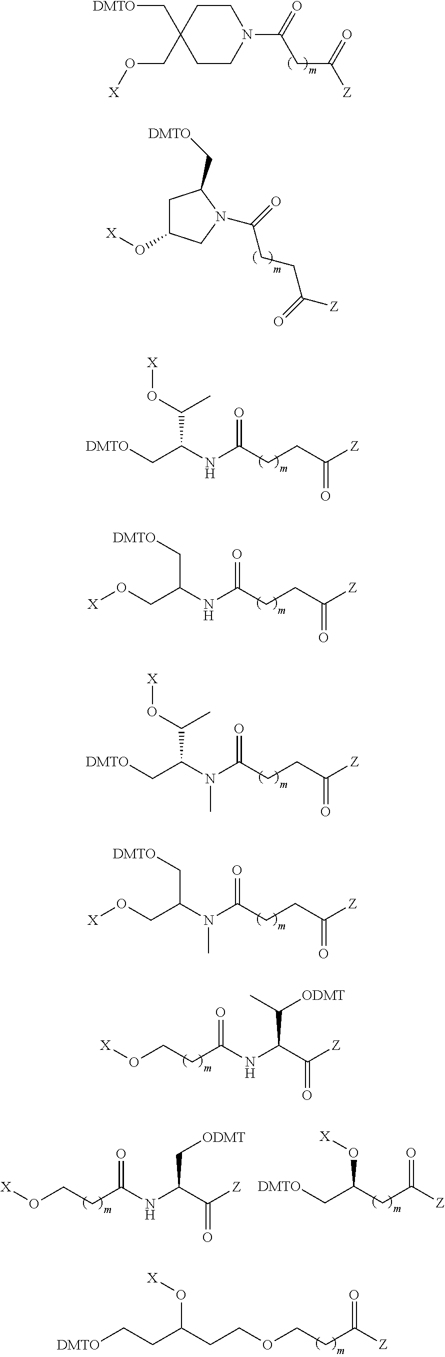

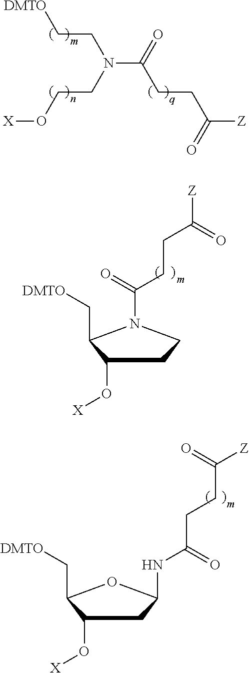

In still other embodiments, where the peptide tag is connected to the scaffold via an amino group, for example its N-terminus, the scaffold is for labeling the terminal position of the oligonucleotide and may be selected from the group consisting of:

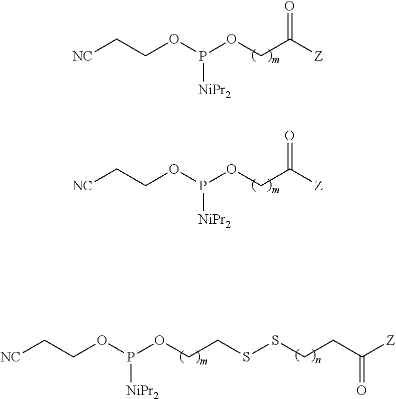

In the above structures, the linkers are shown with DMT (dimethoxytrityl) as a protecting group, which can be interchanged with other suitable protecting groups, including but not limited to MMT (monomethoxytrityl), Trt (trityl), and 2-Cl-Trt (2-chloro-trityl). In these structures, X represents any compatible group that can be effectively coupled onto nucleotide (or nucleotide analogue, e.g. morpholino) chain with solid-phase oligonucleotide synthesis (SPOS) (e.g. phosphoramidite and phosphoramidate), or alternatively, the solid support (e.g. solid support-C(═O)—(CH₂)₂—C(═O)—) of SPOS. Y represents any tag motif, including but not limited to the instances shown below.

In the above structures
m is 0-8
n is 0-8
P¹ is any protecting group on N4 of cytosine (e.g. dmf (N,N-dimethylformamide), Bz (benzyl), Ac (acetyl)) suitable for use in SPOS;
P² is any protecting group on N6 of adenine (e.g. Bz, Pac (phenoxyacetyl)) suitable for use in SPOS;
P³ is any protecting group on N2 of guanine (e.g. iBu (isobutyryl), dmf, iPr-Pac (isopropylphenoxyacetyl)) suitable for use in SPOS;
P⁴ is any protecting group on 2′-O of pentose sugar (e.g. TOM (2-O-triisopropylsilyloxymethyl), TBDMS (2′-O-tert-butyldimethylsilyl), Ac) suitable for use in SPOS of RNA;
P⁵ is any protecting group (e.g. Bz) suitable for use in SPOS of UNA.

In these structures Z is the peptide tag, linked via its N-terminus, and may be selected from the following:

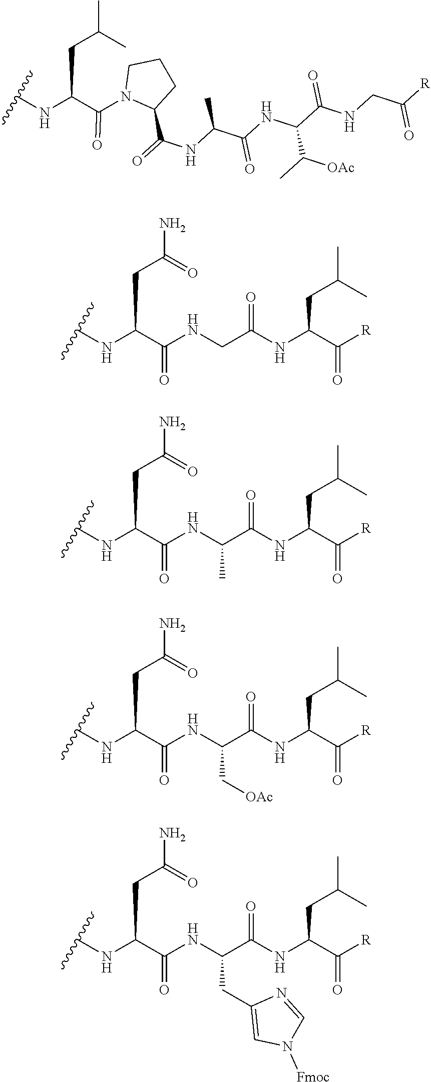

In these structures R is NH₂ (amidated C-terminus) or O-P⁶, with P⁶ being any suitable carboxylic acid protecting group suitable for use in SPOS, such as OMe (methoxy), OtBu (tert-butoxy) and OTrt (trityloxy).

In various embodiments of the methods described herein, ligation of the cargo molecule and the poly(peptide) with a peptide ligase is via a bifunctional adapter comprising at least two ligation motifs for a peptide ligase that can be the same or different. In various embodiments these at least two binding and ligation sites are different and are bound by different ligases. The bifunctional adapter may comprise two peptides with free C-termini that are linked via their side chains and/or N-termini. A suitable adapter is compound (4) in Scheme S5 below.

In another aspect, the present invention also relates to the conjugates obtainable according to any one of the methods of the invention.

### BRIEF DESCRIPTION OF THE DRAWINGS

FIG. 1. Schematic illustration of a phosphoramidite tag-based approach to the enzymatic ligation of single or multiple peptide(s) with an oligonucleotide. (a) Coupling of a phosphoramidite tag onto an oligonucleotide chain (grey dobon) by solid-phase oligonucleotide synthesis, followed by enzymatic ligation of a peptide counterpart (light grey ribbon) containing the cognate ligation handle with the tag-labeled oligonucleotide to produce the desired peptide-oligonucleotide conjugate. (b) Modular coupling of two distinct phosphoramidite tags (1 and 2) onto an oligonucleotide enables positional control on subsequent ligation of two peptides containing the matching ligation handles by the cognate ligases.

FIG. 2. a) Schematic illustration of ligase-assisted ligation between a tag-labeled oligonucleotide and a protein containing the cognate ligation handle. b) SDS-polyacrylamide gel electrophoresis of the crude ligation mixture for sortase-assisted ligation of ODN1 with CFP_{SORT}. CFP_{SORT} was ligated with ODN1 to produce the POC in the presence of the cognate enzyme sortase.

FIG. 3. Both phosphoramidite tag 1 and 2 can be incorporated at either terminal or internal positions. Incorporation of multiple or a mixture of phosphoramidite tag is also possible.

FIG. 4. Simplified schematic for incorporating of tag on N-terminal of a peptide and ligation reaction with cargo molecule using (a) sortase or (b) OaAEP1. Cargo molecule can be dye, drugs, oligonucleotide, aptamer, peptide, protein or antibody.

FIG. 5. Schematic of bifunctional adapter. Adapter allow access of difficult chimeric biomolecule such as N-terminal conjugated protein-oligonucleotide conjugates.

### DETAILED DESCRIPTION

The present invention is based on the inventors' finding that peptide-tagged phosphoramidite units can be readily incorporated into oligonucleotides and thus provide those with a functional moiety (the peptide tag) that allows ligation to a (poly)peptide of choice. This principle can be used for other non-peptide cargo molecules that could be tagged with such short peptides that serve as a ligation motif or ligation handle.

The term “ligation motif”, as used herein, relates to the peptide sequence that is recognized and cleaved by the ligase, for example the N/D-containing peptide motif for PALs, including NGL, NAL, NSL or NHL, or the LPXTG-containing motifs for sortase. The N/D-containing motif is cleaved by the PALs such that all amino acids C-terminal to the N/D are cleaved of and the C-terminus of the N/D residue is then ligated to the N-terminus of another (poly)peptide. The LPXTG-motif is cleaved C-terminal to the T residue and then ligated to the N-terminus of an (poly)peptide that has a C-terminal G residue. The term “ligation motif”, as used herein in relation to ligases such as sortase and PALs, thus typically relates to a peptide sequence the C-terminus of which is, in the ligation reaction, ligated to the N-terminus of another (poly)peptide.

The term “ligation handle” relates to the peptide sequence on the N-terminus of a given (poly)peptide that is linked to the C-terminus of the cleaved ligation motif. The term “ligation handle”, as used herein in relation to ligases such as sortase and PALs, thus typically relates to a peptide sequence with a free N-terminus that is ligated by its N-terminus to the C-terminus of another (poly)peptide.

The term “(poly)peptide”, as used herein, refers to peptides and polypeptides. “Polypeptide”, as used herein, relates to polymers made from amino acids connected by peptide bonds. The polypeptides, as defined herein, can comprise more than 50 amino acids, preferably 100 or more amino acids. “Peptides”, as used herein, relates to polymers made from amino acids connected by peptide bonds. The peptides, as defined herein, can comprise 2 or more amino acids, preferably 5 or more amino acids, more preferably 10 or more amino acids, for example 10 to 50 amino acids. In various embodiments, the term “peptide” relates to peptides with up to 50 amino acids and the term “polypeptide” relates to peptides with more than 50 amino acids.

The methods described herein allow the enzymatic ligation of a (poly)peptide with a cargo molecule. The cargo molecule may be any non-peptide moiety and includes dyes, various pharmaceutically active organic compounds, in particular small molecules, aptamers and oligonucleotides. The methods are herein described by reference to the use of oligonucleotides as the cargo molecule, but it is understood that they are not limited to such applications and can be readily used for the ligation of other cargo molecule types.

The term “oligonucleotides” as used herein refers to oligomers and polymers of nucleotides and analogues thereof. In various embodiments, the term “oligonucleotide” thus also covers “polynucleotides” as well as any analogues or variants thereof, in particular those described herein. Such oligonucleotides may comprise 3 and more nucleotides, typically 10 to 100 nucleotides. The length may however depend on the intended purpose. If the nucleotides are to be used as identifier tags, length of up to 100 nucleotides may typically be sufficient. The length may generally be in the range of 10 to 1000 nucleotides, preferably 10 to 500 or 10 to 100 nucleotides. In various embodiments, the length is 10 to 50, 10 to 30 or 10 to 25 or 12 to 20 nucleotides. In the examples, oligonucleotides of 10 to 18 nucleotides in length are used. The oligonucleotide may be DNA, RNA or any variant thereof. DNA and RNA may both comprise nucleotide variants and analogues. It is also possible to have oligonucleotides that comprise both RNA and DNA nucleotides. The nucleotide variants/analogues may have modified bases, modified sugars, phosphorothioate linkages, phosphorodiamidate rnorpholino unit, locked nucleic acid monomers or any combinations thereof. Specific examples are locked nucleic acid monomers that are available for all common nucleotides, including A, G, T, U and C. Further modified nucleotides (sugar-modified) include 2′-O-methyl or 2′-O-methoxyethyl-modified nucleotides, constrained ethyl (cEt) nucleotides, tricyclo-DNA (tcDNA) nucleotides, nucleotides with 2′-fluoro modifcations, and nucleotides with modified bases/sugars, such as, without limitation, 2-aminopurine, 5-bromo dU, 2′-deoxyuridine, 2,6-diaminopurine, dideoxycytidine, 2′-deoxyinosine, hydroxymethyl dC, inverted dT, iso-dG, iso-dC, inverted dideoxy T, 5-methyl dC, 5-nitroindole, 5-hydroxybutyl-2′-deoxyuridine, 8-aza-7-deazaguanosine or 8-aza-7-deaza-adenosine.

The methods of the invention comprise a steps of: (i) providing at least one cargo molecule modified with a peptide tag and at least one poly(peptide) to be ligated to the cargo molecule, wherein the peptide tag and/or the (poly)peptide comprises a ligation motif for a peptide ligase.

The peptide tag comprises or may even consist of the ligation motif or ligation handle as defined above. It can be advantageous to keep the peptide tag as short as possible to facilitate ligation, as this saves efforts in the synthesis thereof and also prevents unnecessary additional sequences that will be present in the ligated (poly)peptide-cargo molecule/oligonucleotide conjugate. The peptide tag can therefore be, in various embodiments, be up to 20 amino acids in length, preferably up to 18, up to 16, up to 14,m up to 12, or up to 10 amino acids. In some embodiments, it is 2 to 7 amino acids in length. If the peptide tag is the ligation handle it is typically shorter and comprises only 2 to 4, for example 2 or 3 amino acids. If it is the ligation motif it is slightly longer, typically 3 amino acids in the case of PALs or 5 amino acids in the case of sortase. The ligation motif peptide tag length is thus typically 3 to 7 or 3 to 5 amino acids.

The (poly)peptide to be ligated is typically a peptide or polypeptide that has a certain, for example biological, functionality and thus, in various embodiments, has a length of at least 10 or at least 15 or at least 20 amino acids. Generally, the length can be up to thousands of amino acids, such as 1000 amino amino acids, but in various embodiments it has a length of about 15 to 500 amino acids.

The (poly)peptide may be any type of peptide or polypeptide and includes, without limitation, peptide hormones, enzymes, cytotoxic peptides, antibodies, antibody-like polypeptides (antibody mimetics) and antibody fragments, marker proteins, for example fluorescent proteins, peptide aptamers, and other therapeutic proteins. Specific examples include, without limitation, glucagon-like peptide 1 (GLP-1) and RGD-containing peptides.

In the methods described herein, the (poly)peptides to be ligated may be further conjugated to an organic moiety. For this purpose, the (poly)peptide may comprise a reactive group, typically not at the terminus to be ligated. Said reactive group, which may also be a side chain of an amino acid, may then be conjugated to an organic moiety of interest in a further step of the method. The organic moiety may be any molecule or group and comprises pharmaceutically active agents and detectable markers, such as fluorescent markers or biotin. In various embodiments, the active agent may be a small organic molecule pharmaceutical, such as a cancer therapeutic agent, including, but not limited to an anthracycline, such as doxorubicin.

The (poly)peptides to be ligated or cyclized according to the methods and uses disclosed herein can be fusion peptides or polypeptides in which an Asx-containing tag has been C-terminally fused to the (poly)peptide of interest that is to be ligated or fused. The Asx-containing tag preferably has the amino acid sequences of the binding and ligation site for asparaginyl ligases defined herein. Generally, polypeptides and proteins that may be ligated to peptide tags, such as peptide tags of a cargo molecule that also carries signaling or detectable moieties, include, without limitation those described above.

In various embodiments, the ligase activity is used to fuse a peptide bearing a detectable moiety, such as a fluorescent group, including fluoresceins, such as fluorescein isothiocyanate (FITC), or coumarins, such as 7-amino-4-methylcoumarin, to a polypeptide or protein, such as those mentioned above. In various embodiments, the protein can be an antibody fragment or an antibody mimetic.

Detectable markers useful in the methods and uses of the invention include fluorescein or derivatives thereof and/or a peptide that can easily be radiolabeled with elements I-125 or I-131, since this allows using a single reagent imaging of tumors in vivo using PET or SPECT followed by fluorescent detection in organ sections or biopsies.

If the ligation motif is comprised in the (poly)peptide to be ligated, the peptide tag on the oligonucleotide allows the ligation thereto and vice versa. In various embodiments, the (poly)peptide to be ligated comprises the ligation motif, typically at the C-terminus or close to the C-terminus, as the part C-terminal to the cleavage site will be cleaved off. In such embodiments, the peptide tag on the oligonucleotide comprises or consist of the corresponding ligation handle. In such embodiments, the peptide tag on the oligonucleotide may have a free and accessible N-terminus, as this is used for the ligation to the C-terminus of the (poly)peptide. This may mean that the peptide tag is coupled to the oligonucleotide via its C-terminus. Alternatively, the ligation motif may be comprised in the peptide tag. In such embodiments, the (poly)peptide to be ligated has a terminus, typically N-terminus, that allows the ligation to the C-terminus of the cleaved ligation motif. This may mean that the (poly)peptide to be ligated comprises the ligation handle on its N-terminus. Said ligation handle may be part of its natural sequence. In such embodiments, the peptide tag is typically coupled to the oligonucleotide via its N-terminus to provide a free and accessible C-terminus.

The bonding of the peptide tag to the oligonucleotide may be facilitated by a scaffold that is either provided on the peptide tag and allows incorporation into an oligonucleotide, either on the termini of the oligonucleotide, i.e. its 3′ or 5′ terminus, or internally, for example by incorporation into the nascent oligonucleotide. Generally, the peptide tag may be located on either the 5′, the 3′-terminus or internally of the oligonucleotide.

As, in some embodiments, a single oligonucleotide may comprise more than one peptide tag, these may then be located on both ends, on one end and internally or all internally.

One highly suitable scaffold used herein that facilitates easy incorporation to an oligonucleotide either on its ends or internally is a phosphoramidite group containing scaffold. The scaffold may be a reduced amino acid, such as threoninol, serinol, 4-hydroxyprolinol, N-methylserinol and N-methylthreoninol, coupled to the phosphoramidite group via an hydroxy group. Preferred are amino acid variants that comprise two hydroxyl groups, with one preferably being a primary hydroxy group (typically the reduced carboxyl group) and the other being a secondary hydroxyl group. These two type of hydroxyl groups allow the selective protection of the primary hydroxyl group, in particular with a DMT (4,4′-dimethoxytrityl) group which has preference for primary hydroxyl groups.

Generally, a preferred phosphoramidite group used in the methods of the present invention has the formula:

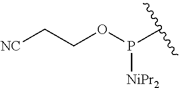

It is understood that the 2-ethylcyanoethyl group that is base labile and protects the phosphite group may be replaced by any other suitable protecting group. Similarly, the isopropyl groups may be replaced by other suitable alkyl groups.

Possible alternatives for such phosphoramidites comprise phosphoramidates, which may have the formula:

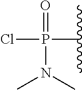 [SMILES: *P(=O)(Cl)N(C)C]

In both formulas, the wavy line indicates the attachment point to the rest of the scaffold or the peptide tag. Both of the above groups may be attached to the rest of the scaffold or the peptide tag by means of an —O— group, for example derived from a hydroxyl group. If the attachment is to the rest of a scaffold, the scaffold may further comprise an amino group or carboxyl group that is then reacted with the C- or N-terminus of the peptide tag to form a peptide bond.

Suitable phosphoramidite group containing scaffolds include, without limitation,

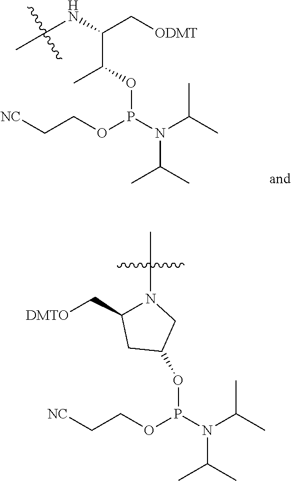

wherein DMT is the protecting group and the wavy line indicates the attachment to the C-terminus of the peptide tag, which may have the sequence GGG, AAA, GL, GG, RL, AL, PL, HV. In these embodiments, the peptide tag is preferably the ligation handle, since the all of the afore-mentioned peptide tags are linked to the oligonucleotide via their C-terminus and thus have a free N-terminus. GGG and AAA are preferred ligation handles for sortase, while GL, RL, PL, HV and GG are preferred ligation handles for PALs.

More specific examples for suitable scaffolds are described in the examples and further comprise the exemplary compounds disclosed below.

In various embodiments, where the peptide tag is connected to the scaffold via a carbonyl group, for example its C-terminus, the scaffold may be selected from the group consisting of:

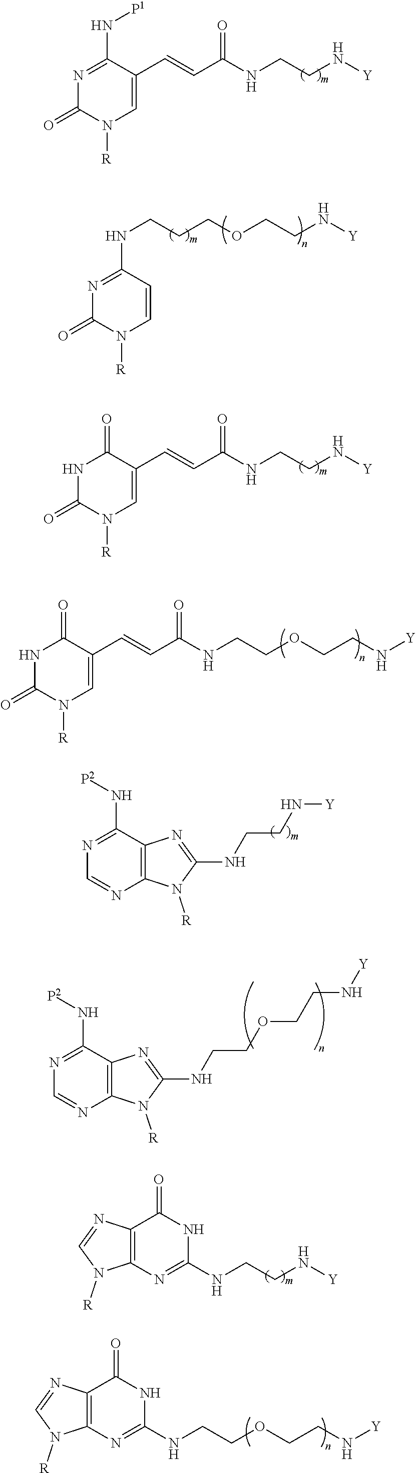

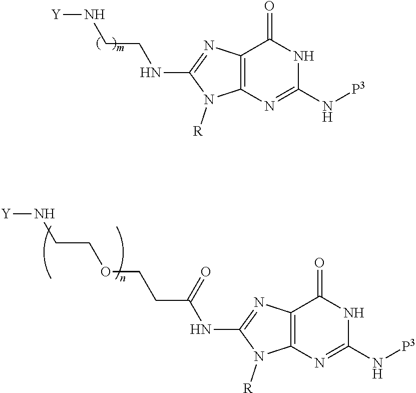

In these embodiments, Y represents the peptide tag that is linked via its C-terminus or a side chain carboxyl group to said nucleobases and may also comprise one linker amino acid analog, such as those mentioned above. In these formulae, R represents the sugar-phosphate part of the nucleotide, preferably a sugar-phosphoramidite or -phosphoramidate part. In such embodiments, R may be selected from

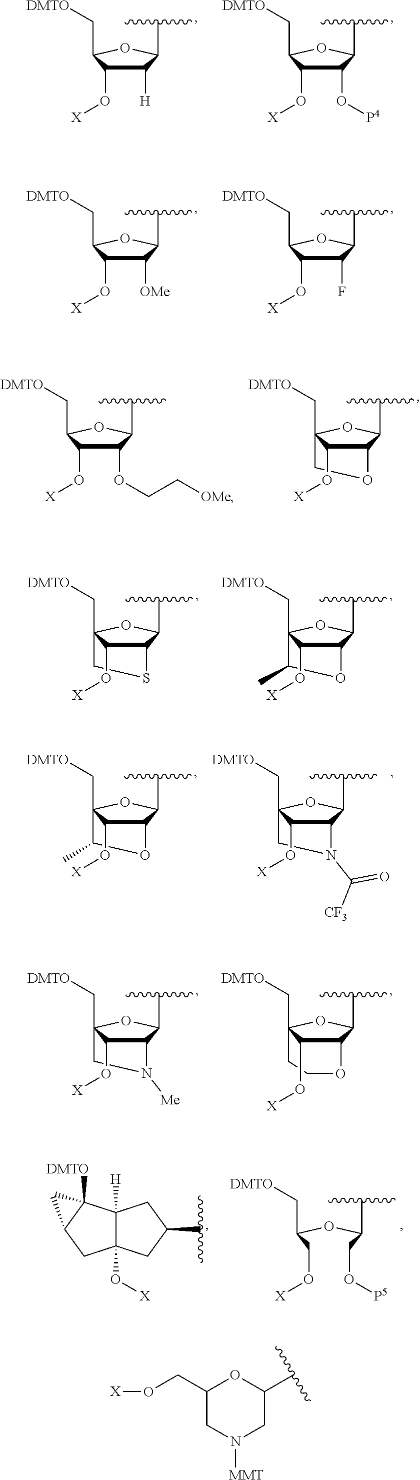

In these embodiments, X is preferably the phosphoramidite or phosphoramidate group, preferably of the structures shown above. Generally, X represents any compatible group that can be effectively coupled to the termini of an oligonucleotide (or oligonucleotide analogue, e.g. morpholino) chain or incorporated into the nascent chain, optionally via solid-phase oligonucleotide synthesis (SPOS), or, alternatively, the solid support (e.g. solid support-C(═O)—(CH₂)₂—C(═O)—) of SPOS.

In the afore-mentioned compounds, the peptide tag is coupled to the nucleotide building block via the nucleobase. However, it may similarly be coupled to the sugar, as exemplarity shown in the following structures:

In various other embodiments, where the peptide tag is connected to the scaffold via a carbonyl group, for example its C-terminus, the scaffold may be selected from the group consisting of:

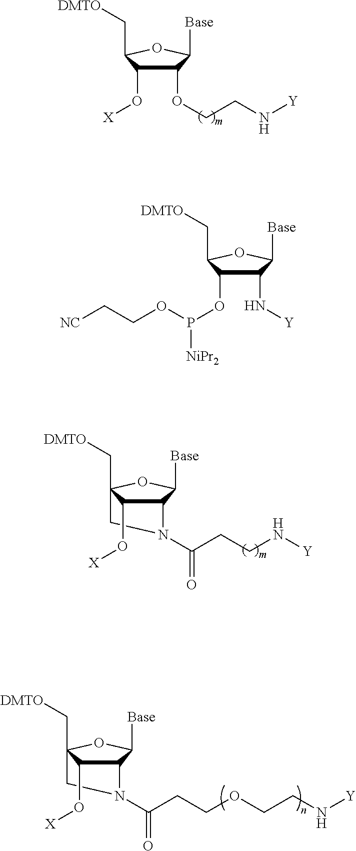

In these formulae, X has the same meaning as described above.

In still other embodiments, where the peptide tag is connected to the scaffold via a carbonyl group, for example its C-terminus, the coupling may be via the backbone and the scaffold may be selected from the group consisting of:

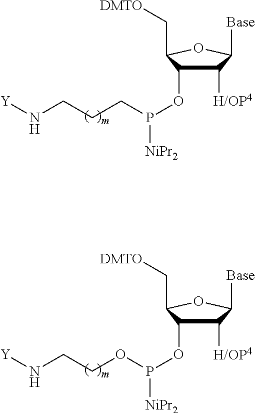

In still other embodiments, where the peptide tag is connected to the scaffold via a carbonyl group, for example its C-terminus, the scaffold is not a nucleotide analogue comprising a base, a sugar and a backbone part, but rather a chemically different moiety. These conjugates are suitable for labeling any position of the oligonucleotide, i.e. may be attached to the termini or incorporated into the nascent oligonucleotide chain, and may be selected from the group consisting of:

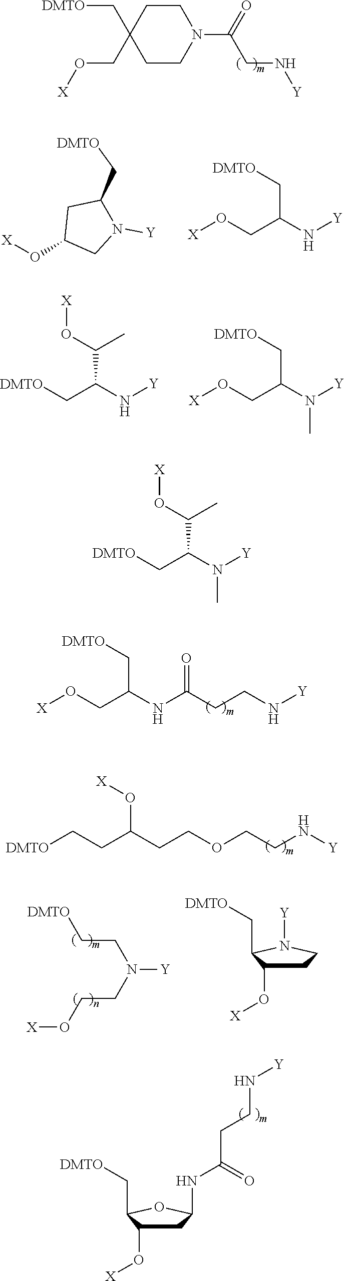

In these formulae, X has the same meaning as described above.

In still other embodiments, where the peptide tag is connected to the scaffold via a carbonyl group, for example its C-terminus, the scaffold is not a nucleotide analogue comprising a base, a sugar and a backbone part, but rather a chemically different moiety, such as a phosphoramidite-linker moiety. These conjugates are suitable for labeling the termini of the oligonucleotide and may be selected from the group consisting of:

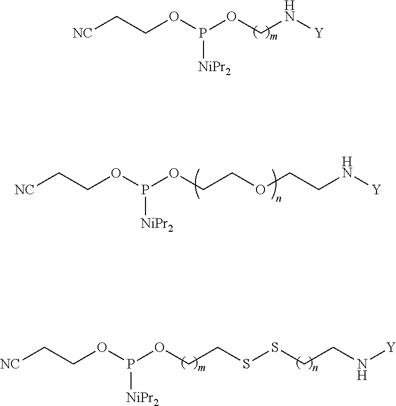

In the above structures Y is the peptide tag, linked via its C-terminus or a side chain carboxylic acid/carboxylate group, and may be selected from the following (the wavy line indicates the attachment to the rest of the structure):

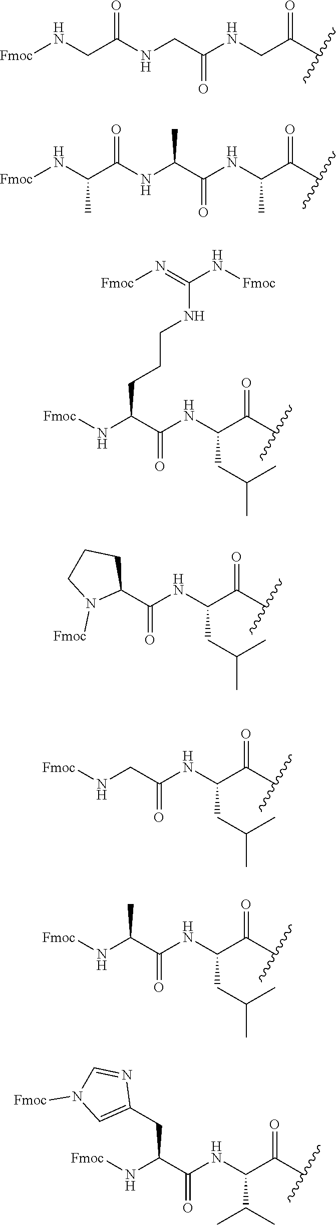

These are embodiments, where Y represents the ligation handle, i.e. is the C-terminal part of the ligation ligated via its N-terminus.

In various alternative embodiments, where the peptide tag is connected to the scaffold via an amino group, for example its N-terminus, the scaffold may be designed such that it allows coupling to the amino group. In such instances, suitable nucleobase structures for attachment may be selected from the group consisting of:

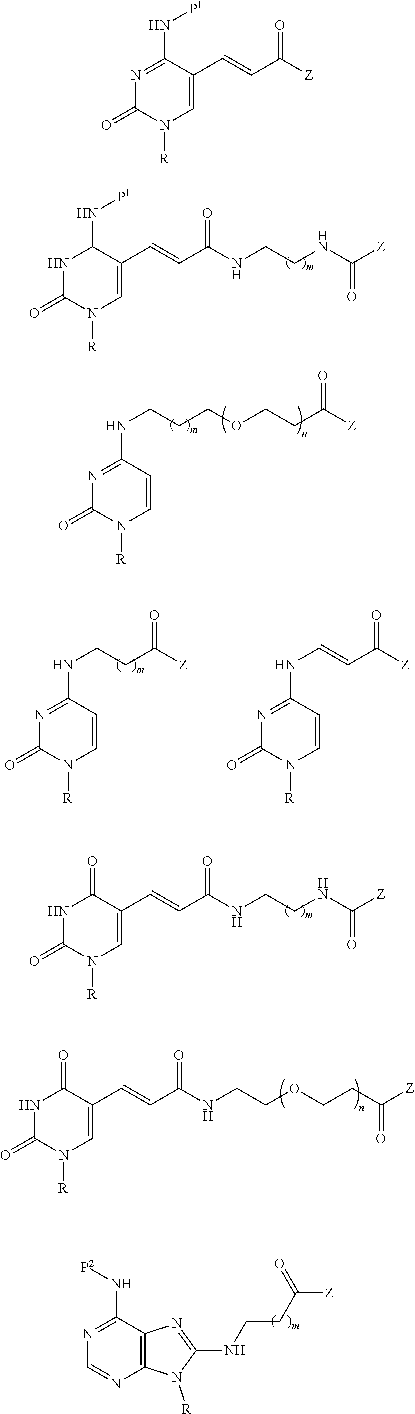

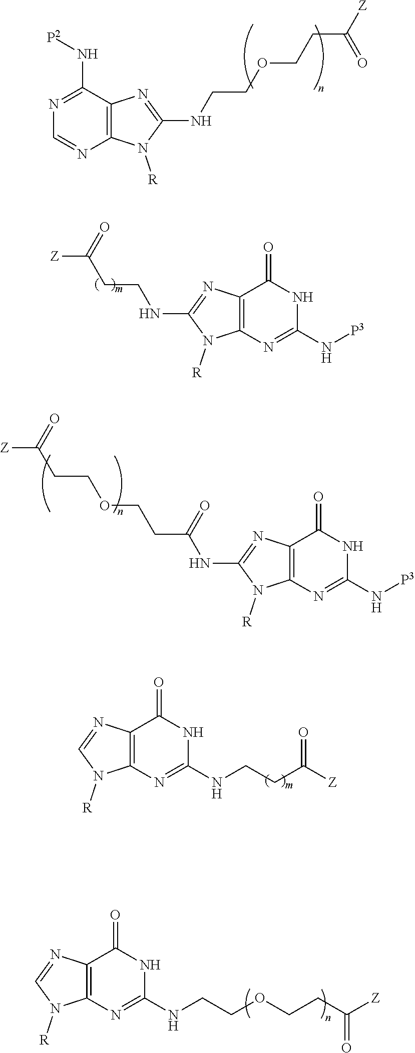

In all these structures, Z represent the peptide tag that is linked via its N-terminus or a side chain amino group to said nucleobases and may also comprise one linker amino acid analog, such as those mentioned above. In these formulae, R represents the sugar-phosphate part of the nucleotide, preferably a sugar-phosphoramidite or -phosphoramidate part. In such embodiments, R may be selected from those already disclosed above.

In various other embodiments, where the peptide tag is connected to the scaffold via an amino group, for example its N-terminus, it may be coupled to the sugar moiety. In various such embodiments, the scaffold may be selected from the group consisting of:

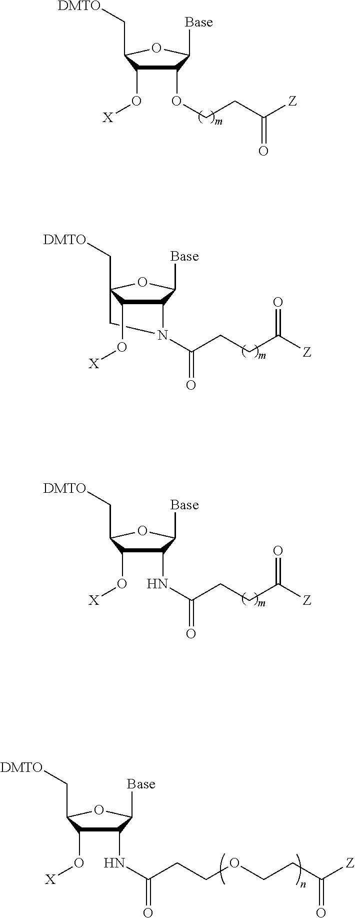

Z has the meaning as defined above. “Base” indicates any nucleobase, such as adenine, guanine, cytosine, thymine and uracil. X has the same meaning as described above.

In still other embodiments, where the peptide tag is connected to the scaffold via an amino group, for example its N-terminus, and specifically to the phosphoramidite, the scaffold may be selected from the group consisting of:

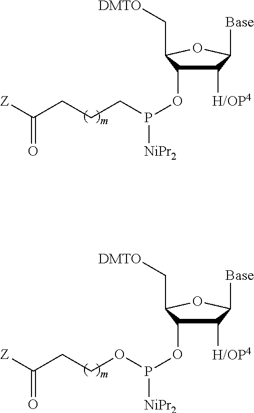

“Base” and Z have the above-described meaning.

In still other embodiments, where the peptide tag is connected to the scaffold via an amino group, for example its N-terminus, the scaffold is not a nucleotide analogue comprising a base, a sugar and a backbone part, but rather a chemically different moiety. These conjugates are suitable for labeling any position of the oligonucleotide, i.e. may be attached to the termini or incorporated into the nascent oligonucleotide chain, and may be selected from the group consisting of:

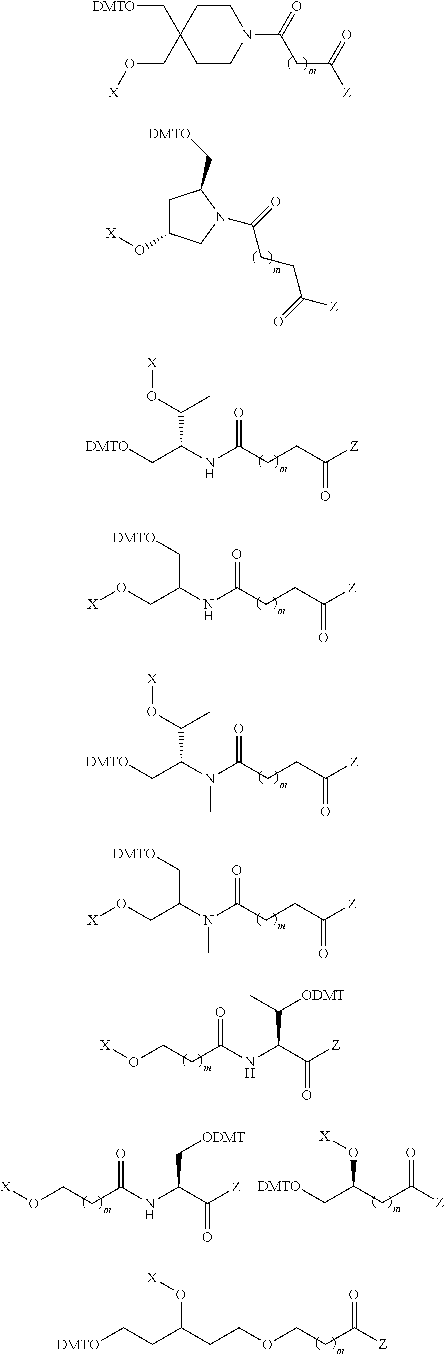

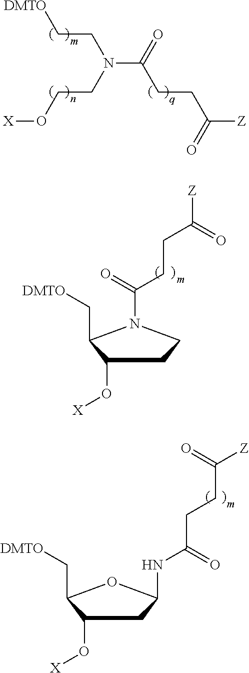

Here, Z and X have the meaning as indicated above.

In still other embodiments, where the peptide tag is connected to the scaffold via an amino group, for example its N-terminus, the scaffold is for labeling the terminal position of the oligonucleotide and may be selected from the group consisting of:

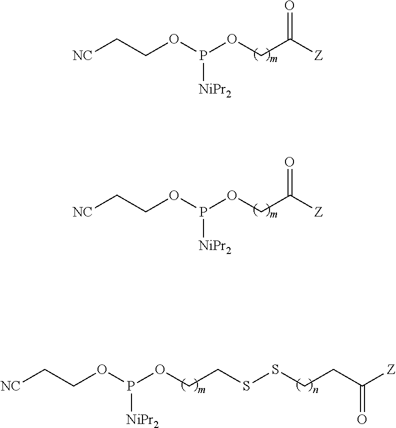

Here, Z has the above-described meaning.

In these structures Z is the peptide tag, linked via its N-terminus or a side chain amino group, and may be selected from the following:

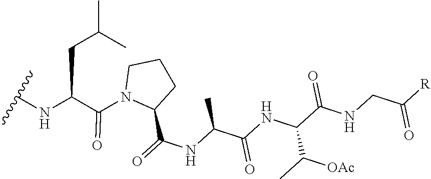

Alternatively, the sortase peptide tag may be LPETG (SEQ ID NO:6).

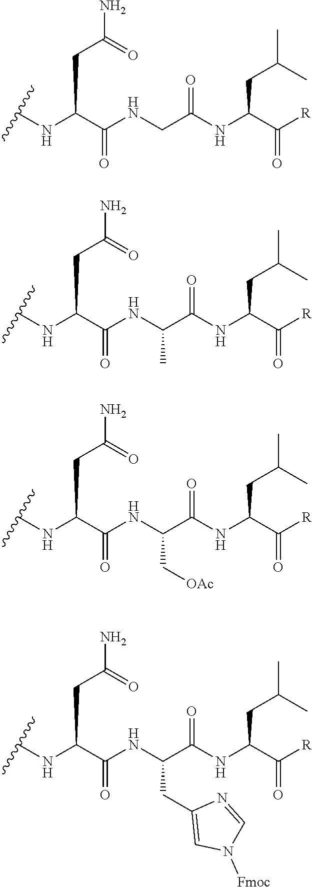

In these structures R is NH₂ (amidated C-terminus) or O-P⁶, with P⁶ being any suitable carboxylic acid protecting group suitable for use in SPOS, such as OMe (methoxy), OtBu (tert-butoxy) and OTrt (trityloxy).

In the above structures, some compounds are shown with a DMT (dimethoxytrityl) protecting group that protects —OH groups from undesired modification. These protecting groups may readily be exchanged for other suitable protecting groups, including but riot limited to MMT (moriomethoxytrityl), Trt (trityl), and 2-Cl-Trt (2-chloro-trityl). In all of these structures. Y represents any peptide tag, including but not limited to the specific ones shown below.

In the above structures
m is 0-8, for example 0, 1, 2, 3, 4, 5, 6, 7 or 8, for example 0 or 1-4, or 0-2; and
n is 0-8, , for example 0, 1, 2, 3, 4, 5, 6, 7 or 8, for example 0 or 1-4, or 0-2.

P¹ to P⁵ are protecting groups for labile groups that shall be protected from undesired side reactions during synthesis of the oligonucleotide with the peptide tag. P′ is any protecting group on N4 of cytosine (e.g. dmf (N,N-dimethylformamide), Bz (benzyl), Ac (acetyl)) suitable for use in SPOS. P² is any protecting group on N6 of adenine (e.g. Bz, Pac (phenoxyacetyl)) suitable for use in SPOS. P³ is any protecting group on N2 of guanine (e.g. iBu (isobutyryl), dmf, iPr-Pac (isopropylphenoxyacetyl)) suitable for use in SPOS. P⁴ is any protecting group on 2′-O of pentose sugar (e.g. TOM (2-O-triisopropylsilyloxymethyl), TBDMS (2′-O-tert-butyldimethylsilyl), Ac) suitable for use in SPOS of RNA. P⁵ is any protecting group (e.g. Bz) suitable for use in SPOS of UNA (unlocked nucleic acid; acyclic analogue of RNA).

The oligonucleotide modified with a peptide tag used in the methods of the invention is obtained by reacting the peptide tag conjugated to a scaffold comprising a reactive group that can be coupled to a nucleotide or nucleotide analogue, preferably a phosphoramidite or phosphoramidate group, with the oligonucleotide under conditions that allow coupling of said reactive group with the oligonucleotide to yield the oligonucleotide modified with a peptide tag. Specific examples for said peptide tag-scaffold molecules that are suitable for coupling to a nucleotide or group that can be incorporated or linked to the oligonucleotide have been disclosed above.

In the methods of the invention, the second step of the method is (ii) contacting the cargo molecule and the poly(peptide) with a peptide ligase that ligates the peptide tag with the (poly)peptide via the ligation motif. As described above, the peptide tag may comprise the ligation handle and the (poly)peptide to be ligated the ligation motif or vice versa. Examples for such motifs have been described herein.

The ligation motif for the peptide ligase may be located at or near the C-terminus of the (poly)peptide to be ligated. This may be advantageous, as it is typically longer than the ligation handle and thus can be added to the (poly)peptide to be ligated by recombinant means so that the accordingly modified (poly)peptide is recombinantly expressed in a host cell. This then allows to keep the peptide tag, which is then effectively the ligation handle, as short as possible and minimizes synthetic efforts.

In various embodiments, the peptide ligase is selected from sortase and peptidyl asparaginyl ligases (PALs; also referred to herein as “asparaginyl ligases”). Suitable PALs have been described in the art, for example WO 2020/226572 A1, and are generally known to those skilled in the art. Examples of such PALs include, but are not limited, to butelase-1, VyPAL2 and OaAEP1b.

The ligases useful according to the present invention exhibit protein ligation activity, i.e. are capable of forming a peptide bond between two amino acid residues, with these two amino acid residues being located on the peptide tag and the (poly)peptide to be ligated. In various embodiments, this protein ligation activity includes an endopeptidase activity, i.e. the peptide bond between two amino acid residues occurs after cleavage of an existing peptide bond.

The asparaginyl ligases may be “Asx-specific” in that the amino acid C-terminal to which ligation occurs, i.e. the C-terminal end of the peptide that is ligated, is either asparagine (Asn or N) or aspartic acid (Asp or D).

The ligases may be naturally occurring enzymes and may be provided in isolated form. “Isolated”, as used herein, relates to the polypeptide in a form where it has been at least partially separated from other cellular components it may naturally occur or associate with. The ligases may be recombinant polypeptides, i.e. polypeptides produced in a genetically engineered organism that does not naturally produce said polypeptide. Both native and recombinant polypeptides may be post-translationally modified by N-linked glycosylation.

In various embodiments, the asparaginyl ligases are selected from butelase-1 (SEQ ID NO:2 and 5), VyPAL2 (SEQ ID NO: 1 and 4) and OaAEP1b (SEQ ID NO:3) and may have the amino acid sequence as set forth in any one of SEQ ID Nos. 1-5 or any functional fragment or variant thereof.

Such variants are at least 80%, preferably at least 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.25%, or at least 99.5% identical to the reference amino acid sequence over their entire length. The variants may also be fragments of the respective reference sequence that retain their activity. Such fragments are typically C- and/or N-terminally truncated versions of the reference sequence and preferably comprise the determinants for the activity of the enzyme as described, for example, in WO 2020/226572 A1.

The identity of nucleic acid sequences or amino acid sequences is generally determined by means of a sequence comparison. This sequence comparison is based on the BLAST algorithm that is established in the existing art and commonly used (cf. for example Altschul et al. (1990) “Basic local alignment search tool”, J. Mol. Biol. 215:403-410, and Altschul et al. (1997): “Gapped BLAST and PSI-BLAST: a new generation of protein database search programs”; Nucleic Acids Res., 25, p. 3389-3402) and is effected in principle by mutually associating similar successions of nucleotides or amino acids in the nucleic acid sequences and amino acid sequences, respectively. A tabular association of the relevant positions is referred to as an “alignment.” Sequence comparisons (alignments), in particular multiple sequence comparisons, are commonly prepared using computer programs which are available and known to those skilled in the art.

A comparison of this kind also allows a statement as to the similarity to one another of the sequences that are being compared. This is usually indicated as a percentage identity, i.e. the proportion of identical amino acid residues at the same positions or at positions corresponding to one another in an alignment. Indications of identity can be encountered over entire polypeptides or only over individual regions. Identical regions of various amino acid sequences are therefore defined by way of matches in the sequences. Such regions often exhibit identical functions. They can be small, and can encompass only a few amino acids. Small regions of this kind often perform functions that are essential to the overall activity of the protein. It may therefore be useful to refer sequence matches only to individual, and optionally small, regions. Unless otherwise indicated, however, indications of identity herein refer to the full length of the respectively indicated nucleic acid sequence or amino acid sequence.

All amino acid residues are generally referred to herein by reference to their one letter code and, in some instances, their three-letter code. This nomenclature is well known to those skilled in the art and used herein as understood in the field.

In the methods and uses described herein, the enzyme, i.e. the ligases, on the one hand and the substrates, i.e. the cargo molecules with the peptide tag(s) and the (poly)peptide to be ligated, on the other hand, can be used in a molar ratio of 1:100 or higher, preferably 1:400 or higher, more preferably at least 1:1000. The reaction is typically carried out in a suitable buffer system at a temperature that allows optimal enzyme activity, usually between ambient (20° C.) and 40° C.

In the methods of the present invention the ligases may be immobilized on a solid support. The major advantages of immobilization on a solid support provide site separation and pseudo-dilution to prevent trans-autolytic degradation and enhance stability. Site-separation of immobilized enzymes permits the use of high enzyme concentrations to accelerate ligation reactions to complete in minutes, such as ligation reactions either under one-pot conditions or in a continuous flow-reactor. Suitable support materials include various resins and polymers that are used in chromatography columns and the like. The support may have the form of beads or may be the surface of larger structure, such as a microtiter plate. Immobilization allows for a very easy and simple contacting with the substrate, as well as easy separation of enzyme and substrate after the synthesis. If the polypeptide with the enzymatic function is immobilized on a solid column material, the ligation/cyclization may be a continuous process and/or the substrate/product solution may be cycled over the column.

In various embodiments, the ligase is glycosylated and the immobilization is facilitated by interaction with a carbohydrate-binding moiety, preferably a concanavalin A moiety or variant thereof, covalently linked to the solid support. In such embodiments, the solid support may be an agarose bead.

In various other embodiments, the ligase is biotinylated and the immobilization is facilitated by interaction with a biotin-binding moiety, preferably a streptavidln, avidin or neutravidin moiety or variant thereof, covalently linked to the solid support. Functionalization of the enzyme with the biotin may be achieved using methods known in the art, such as functionalization with a biotin ester with N-hydroxysuccinimide (NHS), such as succinimidyl-6-(biotinamido)hexanoate. In such embodiments, the solid support may be an agarose bead and the biotin-binding moiety may be an avidin variant, such as neutravidin (deglycosylated avidin).

In various other embodiments, the ligase is immobilized on the solid support by reaction of free amino groups in the polypeptide, for example from lysine side chains, with an N-hydroxysuccinimide functional group on the surface of the solid support. The solid support may be agarose beads.

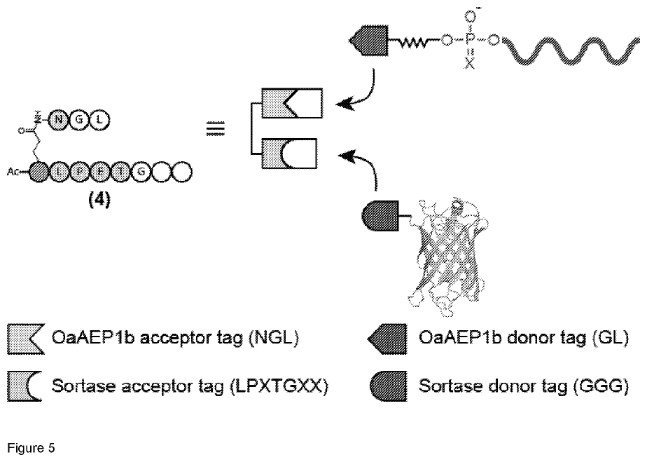

In various embodiments of the methods described herein, ligation of the cargo molecule and the poly(peptide) with a peptide ligase is via a bifunctional adapter comprising at least two ligation motifs for a peptide ligase that can be the same or different. In various embodiments these at least two ligation motifs sites are different and are bound by different ligases. The bifunctional adapter may comprise two peptides or ligation motifs with free C-termini that are linked via their side chains and/or N-termini, optionally to an adapter scaffold. One ligation motif may comprise or consist of the PAKL ligation motifs described herein, and the other may comprise or consist of a different PAL ligation motif or a sortase ligation motif, all as described herein. In such embodiments, both the oligonucleotide and the (poly)peptide to be ligated comprise a peptide motif that corresponds to a ligation handle and allow its N-terminal coupling to the C-terminal ends of the ligation motifs present in the adapter. A suitable adapter is compound (4) in Scheme S5 below. The principle is generally shown in FIG. 5.

The invention is further directed to the specific ligation products obtainable or obtained according to the methods described herein.

Another aspect of the invention relates the specific peptide tag scaffolds, for example the specific phosphoramidite tags, disclosed herein and the modified oligonucleotides described.

The invention is further illustrated by the following non-limiting examples and the appended claims.

### EXAMPLES

### General Methods

Reagents and solvents were purchased from commercial sources (Sigma Aldrich, Acros, Merck, Alfa Aesar). DNA phosphoramidite monomers and thio-modifier C6 S—S phosphoramidite were purchased from Glen Research. Fmoc protected amino acids were purchased from GL Biochem. All reagents were used without further purification. Flash column chromatography was performed using Grace Davisil chromatographic silica media (40-63 μm). Reaction were monitored using Merck TLC silica gel 60 F₂₅₄ aluminium plates. TLC were visualized by UV light, p-anisaldehyde stain or ninhydrin stain. NMR spectra were recorded on a Bruker Avance 300 spectrometer. HRMS analysis were performed on a Waters Q-tof Premier MS.

### Example 1: Synthetic Procedure for (Gly-Gly-Gly) Phosphoramidite Tag

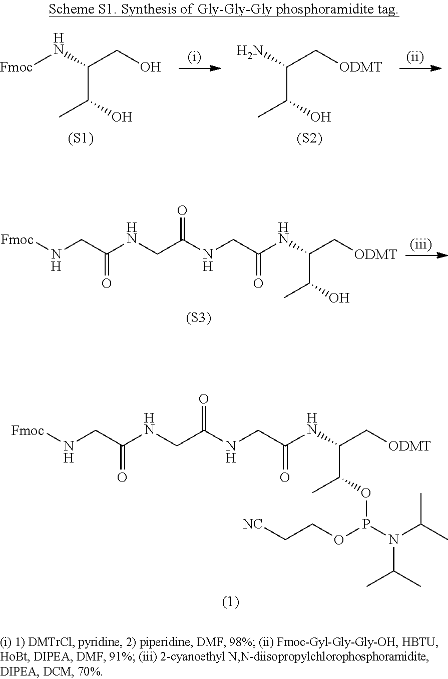

### (2R,3R)-3-amino-4-(bis(4-methoxyphenyl)(phenyl)methoxy)butan-2-ol (S2)

Fmoc-threoninol S1 (300 mg, 916 μmol) was dissolved in anhydrous pyridine (4.00 mL) and added with DMTr-Cl (342 mg, 1.01 mmol) in 3 portions at 10 min interval. The resulting solution was stirred under nitrogen for 5 hours. Solvent was removed under reduced pressure until half volume and diluted with ethyl acetate. The mixture was extracted with saturated NaHCO₃ solution twice followed by brine. The organic phase was dried over Na₂SO₄, filtered and solvent removed under reduced pressure. Anhydrous DMF (1.44 mL) was then added to dissolve the oil. Piperidine (312.1 mg, 0.36 mL, 3.665 mmol) was added slowly and the resulting solution was stirred under nitrogen for 2 h at room temperature. The reaction mixture was diluted with chloroform and extracted with saturated NaHCO₃ solution twice followed by brine. The organic layer was dried over Na₂SO₄, filtered and solvent removed under reduced pressure. The product was purified by flash column chromatography (0-2% MeOH/DCM) to give S2 (366.4 mg, 98.1%) as a yellowish oil. 1H NMR (300 MHz; CDCl₃): δ 1.07 (3H, d, J6.27, Threoninol CH₃), 2.60-2.68 (1H, m, threoninol Hα), 3.08 (1H, dd, J 5.85 9.41 threoninol CH₂), 3.24 (1H, dd, J 4.17 9.41 threoninol CH₂), 3.65 (1H, quintet, threoninol Hβ), 3.77 (6H, s, 2×OCH₃), 6.82 (4H, d, J 8.82, H—Ar), 7.15-7.35 (7H, m, H—Ar), 7.38-7.46 (2H, m, H—Ar). ¹³C NMR (75 MHz, CDCl₃): δ 19.96, 55.26, 57.16, 65.97, 68.04, 86.20, 113.21, 126.88, 127.91, 128.16, 130.08, 136.01, 136.10, 144.90, 158.57. HRMS calc. for C₂₅H₃₀NO₄: 408.2175; found: 408.2174.

(9H-fluoren-9-yl)methyl ((R)-4-((R)-1-hydroxyethyl)-1,1-bis(4-methoxyphenyl)-6,9,12-trioxo-1-phenyl-2-oxa-5,8,11-triazatridecan-13-yl)carbamate (S3)

A solution of Fmoc-Gly-Gly-Gly-OH (505 mg, 1.23 mmol), DIPEA (476 mg, 0.64 mL, 3.68 mmol) , HoBt (166 mg, 1.23 mmol) and HBTU (512 mg, 1.35 mmol) dissolved in anydrous DMF (3.5 mL) was added slowly to a solution of S2 (500 mg, 1.23 mmol) in anhydrous DMF (3 mL). The resulting solution was stirred under nitrogen for 2 h at room temperature. The reaction mixture was poured into ice cold water and extracted twice with ethyl acetate. The combine organic layer was washed with water followed by brine. The organic layer was then dried over Na₂SO₄, filtered and solvent removed under reduced pressure. The product was purified by flash column chromatography (0-6% MeOH/DCM) to afford the product S3 (899.1 mg, 91.5%) as a faint yellowish solid. ¹H NMR (300 MHz; MeOD): δ 1.08 (3H, d, J 6.38, threoninol —CH₃), 3.06-3.18 (1H, m, threoninol CH₂),3.24-3.33 (1H, m, threoninol CH₂), 3.73 (6H, s, 2×OCH₃), 3.82 (2H, s, Gly —CH₂) 3.87-4.03 (5H, m, threoninol Hα and 2×Gly —CH₂) 4.03-4.13 (1H, m, threoninol Hβ), 4.17 (1H, t, J 6.45, Fmoc —CH), 4.36 (2H, d, J 6.76, Fmoc —CH₂), 6.83 (4H, d, J 8.84, H—Ar), 7.14-7.48 (13H, m, H—Ar), 7.57-7.69 (2H, m, H—Ar), 7.79 (2H, d, J 7.50, H—Ar). ¹³ C NMR (75 MHz, MeOD): δ 20.33, 43.46, 43.88, 45.15, 48.29, 55.67, 56.57, 87.32, 114.06, 120.90, 126.18, 127.70, 20 128.15, 128.73, 128.78, 129.28, 131.24, 137.21, 137.29, 142.53, 145.16, 145.19, 146.42, 159.29, 159.99, 171.62, 172.09, 173.14. HRMS calc. for C₄₆H₄₉N₄O₉: 801.3500; found: 801.3503.

### (9H-fluoren-9-yl)methyl((4R)-4-((1R)-1-(((2-cyanoethoxy)(diisopropylamino)phosphaneyhoxy)ethyl)-1,1-bis(4-methoxyphenyl)-6,9,12-trioxo-1-phenyl-2-oxa-5,8,11-triazatridecan-13-yl)carbamate (1)

S3 (346.8 mg, 433.0 μmol) that was suspended in anhydrous DCM (3.34 mL) was added with DIPEA (139.9 mg, 0.19 mL, 1.083 mmol). 2-cyanoethyl N,N-diisopropylchlorophosphoramidite (123.0 mg, 115.9 μL, 519.6 μmol) was then added dropwise over several min and the resulting solution was then allowed to stir at room temperature under nitrogen for 30 min. The reaction mixture was diluted with DCM and extracted with saturated KCI solution. The organic layer was dried over Na₂SO₄, filtered and solvent removed under reduced pressure. The crude product was then purified by flash column chromatography (0-5% MeOH/DCM) to give 1 (306.9 mg, 70.8%) as a white foam. ³¹P NMR (162 MHz; CDCl₃): δ 148.13, 148.82.

### Example 2: Synthetic Procedures for (Gly-Leu) Phosphoramidite Tag

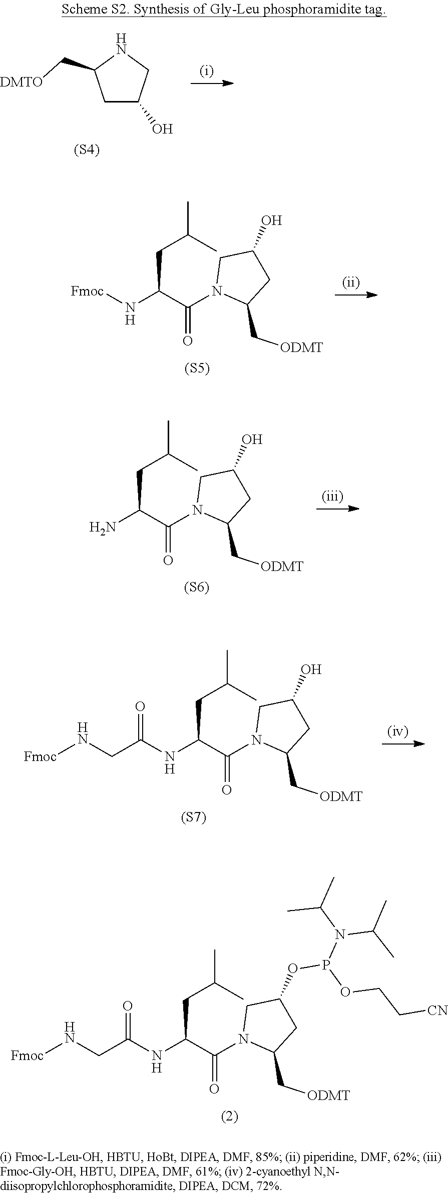

### (3R,5S)-5-((bis(4-methoxyphenyl)(phenyl)methoxy)methyl)pyrrolidin-3-ol (S4)

Compound (S4) was synthesized according to procedure reported by Prakash et al. (Prakash et al. Nucleic Acids Res. 2014, 42 (13), 8796-807).

### (9H-fluoren-9-yl)methyl((S)-1-((2S,4R)-2-((bis(4-methoxyphenyl)(phenyl)methoxy)methyl)-4-hydroxypyrrolidin-1-yl)-4-methyl-1-oxopentan-2-yl)carbamate (S5)

A solution of S4 (200 mg, 477 μmol) in anhydrous DMF (1 mL) was added to a stirring solution of Fmoc-L-Leu-OH (168 mg, 477 limo), DIPEA (185 mg, 0.25 mL, 1.43 mmol), HBTU (199 mg, 524 μmol) and HoBt (64.4 mg, 477 μmol) in anhydrous DMF (1.8 mL). The resulting mixture was stirred under nitrogen for 1 h at room temperature. The reaction mixture was poured into ice cold water and extracted twice with ethyl acetate. The combine organic layer was washed with water followed by brine and dried over Na₂SO₄. Solvent was removed under reduced pressure and the crude product was purified by silica flash column chromatography (30-50% EA/Hex with 1% TEA) to give S5 (304.7 mg, 84.7%) as a white foam. ¹H NMR (300 MHz, CDCl₃): δ 0.81-1.05 (6H, m, Leu 2×CH₃), 1.49-1.60 (2H, m, Leu Hβ), 1.70-1.80 (1H, m, Leu Hγ), 1.91-2.23 (2H, m, prolinol H3′/H3″), 3.07-3.16 (1H, m, prolinol H1′), 3.45-3.60 (1H, m, prolinol H1″), 3.65-3.73 (1H, m, prolinol H5′), 3.78 (6H, s, 2×OCH₃), 3.88-4.10 (1H, m, prolinol H5″), 4.14-4.25 (1H, m, Fmoc CH), 4.27-4.40 (2H, m, Fmoc CH₂), 4.40-4.67 (3H, m, prolinol H2, H4, Leu Hα), 5.52 (1H, d, J 8.52, NH), 6.64-6.88 (4H, m, H—Ar), 7.17-7.43 (13H, m, H—Ar), 7.52-7.63 (2H, m, H—Ar), 7.76 (2H, d, J 7.35, H-Ar). ¹³C NMR (75 MHz; CDCl₃): δ 8.53, 22.11, 23.26, 24.65, 36.45, 42.06, 42.51, 45.63, 47.16, 50.87, 55.20, 55.73, 56.02, 59.97, 63.01, 67.16, 70.49, 86.01, 113.08, 119.95, 125.17, 126.79, 127.08, 127.69, 127.77, 128.09, 129.14, 130.01, 135.95, 136.13, 141.29, 143.74, 143.78, 143.91, 144.96, 158.46, 171.60 . HRMS calc. for C₄₇H₅₀N₂O₇: 755.3696; found: 755.3687

### (S)-2-amino-1-((2S,4R)-2-((bis(4-methoxyphenyl)(phenyl)methoxy)methyl)-4-hydroxypyrrolidin-1-yl)-4-methylpentan-1-one (S6)

To a solution of S5 (369.4 mg, 489.3 μmol) in anhydrous DMF (1.7 mL) was added piperidine (166.7 mg, 193 μL, 1.957 mmol). The resulting solution was stirred at room temperature under nitrogen for 2 h. The reaction mixture was diluted with chloroform and extracted with saturated sodium bicarbonate solution twice followed by brine. The organic layer was dried over Na₂SO₄, filtered and solvent removed under reduced pressure. The crude product was then purified by silica flash column chromatography (0-5% MeOH/DCM with 1% TEA) to give S6 (183.7 mg, 70.5%) as a white foam. 1H NMR (300 MHz; CDCl₃): δ 0.80-0.97 (6H, m, Leu 2×CH₃), 1.41-1.61 (2H, m, Leu Hβ) 1.67-1.87 (1H, m, Leu Hγ), 1.89-2.04 (1 H, m, prolinol H3′), 2.05-2.20 (1H, m, prolinol H3″), 2.96-3.15 (1H, m, prolinol H1′), 3.46-3.64 (2H, m, prolinol H1″, Leu Hα), 3.67-3.82 (8H, m, 2×OCH₃, H5′, H5″,), 3.82-3.96 (2H, m, NH₂), 4.39-4.48 (1H, m, prolinol H2), 4.51 (1H, s, prolinol H4), 6.74-6.86 (4H, m, H—Ar), 7.13-7.30 (7H, m, H—Ar), 7.30-7.38 (2H, d, J 7.68, H—Ar). ¹³C NMR (75 MHz, CDCl₃): δ 22.08, 23.29, 24.67, 36.10, 43.79, 44.76, 50.79, 55.22, 55.28, 55.84, 56.16, 62.87, 70.42, 85.94, 113.10, 126.78, 127.78, 128.11, 130.02, 135.98, 136.19, 145.00, 158.46. HRMS calc. for C₃₂H₄₀N₂O₅: 533.3015; found: 533.3016.

### (9H-fluoren-9-yl)methyl (2-(((S)-1-((2S,4R)-2-((bis(4-methoxyphenyl)(phenyl)methoxy)methyl)-4-hydroxypyrrolidin-1-yl)-4-methyl-1-oxopentan-2-yl)amino)-2-oxoethyl)carbamate (S7)

To a stirring solution of S6 (293.5 mg, 551.0 μmol) in anhydrous DMF (1.6 mL) was added a solution of Fmoc-Gly-OH (163.8 mg, 551.0 μmol), DIPEA (284.9 mg, 0.38 mL, 2.204 mmol) and HBTU (229.9 mg, 606.1 μmol) in anhydrous DMF (1.5 mL). The resulting mixture was stirred under nitrogen for 1 h at room temperature. The reaction mixture was poured into ice cold water and extracted twice with ethyl acetate. Combined organic layer was washed with water followed by brine. The organic layer was dried over Na₂SO₄, filtered and solvent removed under reduced pressure. The crude product was purified by silica flash column chromatography (50-100% EA/Hex with 1% TEA) to give S7 (321.7 mg, 71.9%) as a faint yellowish foam. ¹H NMR (300 MHz; CDCl₃): δ 0.78-1.02 (6H, m, Leu 2×CH₃), 1.46-1.73 (3H, m, Leu Hβ, Leu Hγ), 1.84-2.24 (2H, m, prolinol H3′/H3″), 2.93-3.11 (1H, m, prolinol H1′), 3.51-3.67 (2H, m, prolinol H1″, prolinol H5′), 3.60-3.69 (1H, m, prolinol H5″), 3.71 (6H, s, 2×OCH₃), 3.79-3.91 (2H, m, Gly Hα), 3.96-4.08 (1H, m, prolinol H5″), 4.09-4.24 (1H, m, Fmoc CH), 4.33 (2H, d, J 6.54, Fmoc CH₂), 4.52-4.40 (2H, m, prolinol H2′, prolinol H4′), 4.72-4.92 (1H, m, Leu Hα), 6.18 (1H, s, Gly NH), 6.75 (4H, d, J 8.49, H—Ar) 7.09-7.28 (10H, m, H—Ar and Leu NH), 7.29-7.43 (4H, m, H—Ar), 7.56 (2H, d, J 7.17, H—Ar), 7.72 (2H, d, J 7 .53, H—Ar). ¹³C NMR (75 MHz, CDCl₃): δ 22.19, 23.12, 24.66, 35.98, 42.05, 44.15, 47.06, 49.49, 55.13, 56.04, 56.22, 62.60, 67.20, 70.36, 85.85, 113.02, 119.89, 125.15, 126.73, 127.09, 127.67, 127.72, 128.03, 129.95, 129.99, 135.85, 136.13, 141.22, 143.81, 144.93, 158.40, 169.50, 171.43. HRMS calc. for C₄₉H₅₃N₃O₈: 812.3909; found: 812.3911.

### (2S,4R)-1-((((9H-fluoren-9-yl)methoxy)carbonyl)glycyl-L-leucyl)-2-((bis(4-methoxyphenyl)(phenyl)methoxy)methyl)-4-(((2-cyanoethoxy)(diisopropylamino)phosphaneyhoxy)pyrrolidin-3-ylium (2)

DIPEA (119.1 mg, 0.16 mL, 921.2 μmol) was added to a stirring solution of S7 (299.2 mg, 368.5 μmol) in anhydrous DCM (2.85 mL) under nitrogen. N,N-diisopropylchlorophosphoramidite (104.7 mg, 98.64 μL, 442.2 μmol) was then added dropwise over several min to the reaction mixture and the resulting solution was stirred at room temperature under nitrogen for 30 min. The reaction mixture was diluted with anhydrous DCM and extracted with saturated KCI solution. The organic layer was dried over Na₂SO₄, filtered and solvent removed under reduced pressure. The crude product was purified by silica flash column chromatography (50-65% EA/Hex with 1% TEA) to give 2 (333.2 mg, 89.4%) as a white foam. ³¹P NMR (162 MHz; CDCl₃): δ 147.45, 147.96, 148.16, 148.57.

### Example 3: Synthetic Procedures for (Pro-Leu) Phosphoramidite Tag

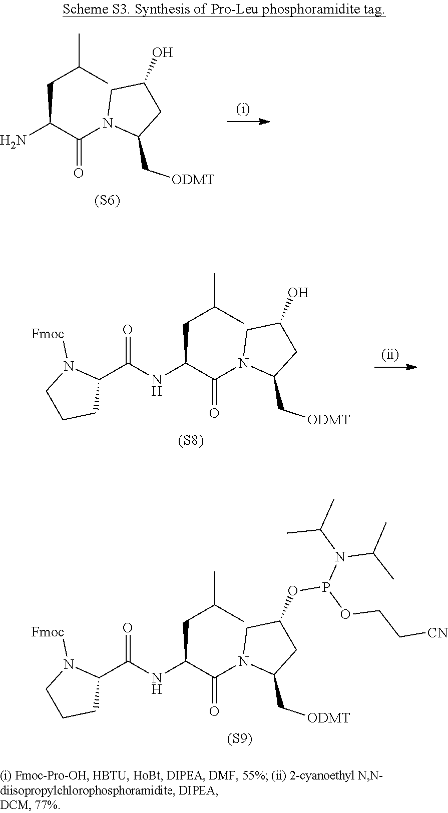

(9H-fluoren-9-yl)methyl (S)-2-(((S)-1-((2S,4R)-2-((bis(4-methoxyphenyl)(phenyl)methoxy)methyl)-4-hydroxypyrrolidin-1-yl)-4-methyl-1-oxopentan-2-yl)carbamoyl)pyrrolidine-1-carboxylate (S8)

A solution of S6 (259.4 mg, 487.0 μmol) in anhydrous DMF (1.7 mL) was added dropwise to a stirring solution of Fmoc-Pro-OH (164.3 mg, 487.0 μmol), DIPEA (188.8 mg, 0.26 mL, 1.461 mmol), HBTU (203.2 mg, 535.8 μmol) and HoBt (65.8 mg, 486.9 μmol) in anhydrous DMF (1.2 mL). The resulting solution was stirred at room temperature under nitrogen for 1.5 h. The reaction mixture was poured into ice cold water and extracted twice with ethyl acetate. Combined organic layer was washed with water followed by brine. The organic layer was dried over Na₂SO₄, filtered and solvent removed under reduced pressure. The crude product was purified by silica flash column chromatography (50-90% EA/Hex with 1% TEA) to give S8 (228.0 mg, 55.0%) as yellow foam.

### (9H-fluoren-9-yl)methyl (2S)-2-(((2S)-1-((2S,4R)-2-((bis(4-methoxyphenyl)(phenyl)methoxy)methyl)-4-(((2-cyanoethoxy)(diisopropylamino)phosphaneyl)oxy)pyrrolidin-1-yl)-4-methyl-1-oxopentan-2-yl)carbamoyl)pyrrolidine-1-carboxylate (S9)

DIPEA (86.5 mg, 0.12 mL, 669.0 μmol) was added to a stirring solution of S8 (228.0 mg, 268.0 μmol) in anhydrous DCM (2.07 mL) under argon. N,N-diisopropylchlorophosphoramidite (76.0 mg, 71.6 μL, 321.0 μmol) was then added dropwise over several min to the reaction mixture and the resulting solution was stirred at room temperature under argon for 30 min. The reaction mixture was diluted with anhydrous DCM and extracted with saturated KCl solution. The organic layer was dried over Na₂SO₄, filtered and solvent removed under reduced pressure. The crude product was purified by silica flash column chromatography (40-60% EA/Hex with 1% TEA) to give S9 (217.8 mg, 77.3%) as a pale-yellow foam. ³¹P NMR (162 MHz; CDCl₃): δ 147.52, 147.75, 147.85, 147.99, 148.03, 148.39.

### Example 4: Synthetic Procedures for Asn(Trt)-Gly-Leu-OtBu Tag

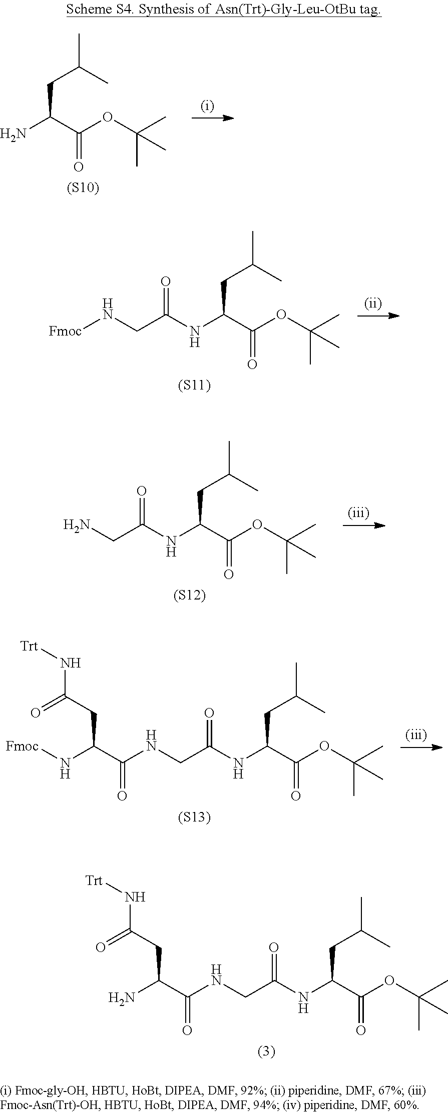

### tert-butyl (((9H-fluoren-9-yl)methoxy)carbonyl)glycyl-L-leucinate (S11)

A solution of Fmoc-Gly-OH (476 mg, 1 Eq, 1.60 mmol), DIPEA (621 mg, 0.84 mL, 3 Eq, 4.81 mmol) , HoBt (216 mg, 1 Eq, 1.60 mmol) and HBTU (668 mg, 1.1 Eq, 1.76 mmol) that was dissolved in anhydrous DMF (1.35 mL) was added to a stirring solution of tert-butyl L-leucinate S10 (300 mg, 1 Eq, 1.60 mmol) dissolved in anhydrous DMF (1 mL). The resulting solution was stirred at room temperature under nitrogen atmosphere for 2 h. The reaction mixture was poured into ice cold water and extracted twice with ethyl acetate. The combine organic layer was then washed with brine. The organic layer was dried over Na₂SO₄, filter and solvent removed under reduced pressure. The crude product was purified by flash column chromatography to afford S11 (684.4 mg, 91.6%) as a white foam. ¹H NMR (300 MHz; CDCl₃): δ 0.85-0.97 (6H, m, 2×CH₃-Leu), 1.44 (9H, s, 3×CH₃-tBu), 1.46-1.75 (3H, m, H_{γ}-Leu and H_{β}-Leu), 3.94 (2H, d, J5.16, CH₂-Gly), 4.17 (1 H, t, J 7.11, CH-Fmoc), 4.37 (2H, d, J 6.95, CH₂-Fmoc), 4.47-4.63 (1H, m, H_{α}-Leu), 5.83 (1H, t, J 5.16, NH-Gly), 6.76 (1H, d, J 7.91, NH-Leu), 7.27 (2H, t, J 7.27, H—Ar), 7.37 (2H, t, J 7.40, H—Ar), 7.57 (2H, d, J 7.38, H—Ar), 7.73 (2H, d, J 7.47, H—Ar). ¹³C NMR (75 MHz, CDCl₃): δ 22.11, 22.81, 24.97, 28.03, 41.77, 44.42, 47.11, 51.48, 67.35, 82.11, 120.02, 125.17, 127.13, 127.76, 141.32, 143.85, 156.6, 18.79, 172.18. HRMS calc. for C₂₇H₃₅N₂O₅: 467.2546; found: 467.2565.

### tert-butyl glycyl-L-leucinate (S12)

To a solution of S11 (684.4 mg, 1 Eq, 1.467 mmol) in DMF (2.32 mL) was added piperidine (499.6 mg, 0.58 mL, 4 Eq, 5.867 mmol). The resulting solution was stirred under nitrogen for 1 h. The reaction mixture was diluted with chloroform and extracted with saturated sodium bicarbonate solution followed by brine. The organic layer was dried over Na₂SO₄, filtered and solvent removed under reduced pressure. The crude product was purified by silica flash column chromatography to give S12 (239.0 mg, 66.68%) as a colourless oil. ¹H NMR (300 MHz; CDCl₃): δ 0.95 (6H, d, J 6.05, 2×CH₃-Leu), 1.47 (9H, s, 3×CH₃-tBu), 1.50-1.77 (3H, m, H_{γ}-Leu and H_{β}-Leu), 1.83 (2H, NH₂-Gly), 3.39 (2H, s, CH₂-Gly), 4.45-4.62 (1H, m, H_{α}-Leu), 7.54 (1H, d, J 8.06, NH-Leu). ¹³C NMR (75 MHz, CDCl₃): δ 22.11, 22.88, 25.01, 28.03, 41.91, 44.64, 50.92, 81.79, 172.34. HRMS calc. for C₁₂H₂₅N₂O₃: 245.1865; found: 245.1855.

### tert-butyl N2-(((9H-fluoren-9-yl)methoxy)carbonyl)-N4-trityl-L-asparaginylglycyl-L-leucinate (S13)

A solution of S12 (221.5 mg, 1 Eq, 906.5 μmol) and DIPEA (351.5 mg, 0.48 mL, 3 Eq, 2.720 mmol) that was dissolved in anhydrous DMF (2 mL) was added with a solution of Fmoc-Asn(Trt)-OH (540.9 mg, 1 Eq, 906.5 μmol), HoBt (122.5 mg, 1 Eq, 906.5 μmol) and HBTU (378.2 mg, 1.1 Eq, 997.2 μmol) dissolved in anhydrous DMF (2.8 mL). The resulting yellow solution was stirred at room temperature under nitrogen atmosphere for 2 h. The reaction mixture was poured into ice cold water and extracted twice with ethyl acetate. The combine organic layer was then washed with brine, dried over Na₂SO₄, filter and solvent removed under reduced pressure. The crude product was purified by flash column chromatography to afford S13 (703 mg, 94.2%) as a white solid. ¹H NMR (300 MHz; CDCl₃): δ 0.85 (6H, dd, J 6.08 2.00, 2×CH₃-Leu), 1.30-1.67 (12H, m, 3×CH₃-tBu, H_{γ}-Leu and H_{β}-Leu), 2.56-2.75 (1 H, m, H_{β}-Asn), 2.94-3.11 (1H, m, H_{β}-Asn), 3.70-3.99 (2H, m, CH₂-Gly), 4.05-4.23 (1 H, m, CH-Fmoc), 4.30-4.57 (4H, m, CH₂-Fmoc, Hα-Leu and Hα-Asn), 6.36 (1H, d, J 6.60, NH-Asn), 6.82 (1H, d, J 7.44, NH-Leu), 7.10-7.32 (19H, m, H—Ar, NH-Gly and NH-Asn side chain), 7.36 (2H, t, J 7.35, H—Ar), 7.56 (2H, d, J 7.35, H—Ar), 7.72 (2H, dd, J 7.38 3.21, H—Ar). ¹³C NMR (75 MHz, CDCl₃): δ 20.20, 22.64, 24.90, 28.30, 38.53, 41.26, 43.17, 47.16, 51.53, 52.19, 67.32, 70.89, 81.81, 120.05, 125.19, 127.18, 127.79, 128.03, 128.71, 141.34, 143.77, 144.28, 168.30, 170.13, 171.29, 172.22.

### tert-butyl N4-trityl-L-asparaginylglycyl-L-leucinate (3)

S13 (703 mg, 1 Eq, 854 μmol) was dissolved in DMF (1.50 mL). The solution was added with piperidine (291 mg, 0.34 mL, 4 Eq, 3.42 mmol) and the resulting solution was stirred under nitrogen for 1 h. The reaction mixture was diluted with chloroform and extracted with saturated sodium bicarbonate solution followed by brine. The organic layer was dried over Na₂SO₄, filtered and solvent removed under reduced pressure. The crude product was purified by silica flash column chromatography to give 3 (308.8 mg, 60.2%) as a white foam. ¹H NMR (400 MHz; CDCl₃): δ 0.88 (6H, dd, J6.44 2.56, 2×CH₃-Leu), 1.32-1.67 (3H, m, H_{γ}-Leu and H_{β}-Leu), 1.41 (9H, s, 3×CH₃-tBu), 1.91 (2H, s, NH₂), 2.62 (2H, d, J 5.88, H_{β}-Asn), 3.60 (1H, t, J5.86, H_{α}-Asn), 3.74-3.96 (2H, m, CH₂-Gly), 4.36-4.45 (1H, m, H_{α}-Leu), 7.00 (1H, d, J8.01, NH-Leu), 7.13-7.28 (15H, m, H—Ar), 7.90-8.05 (2H, m, NH-Gly and NH-Asn side chain). ¹³C NMR (101 MHz, CDCl₃): δ 22.19, 22.67, 24.88, 28.01, 41.40, 41.51, 42.93, 51.47, 52.36, 70.49, 81.76, 126.92, 127.87, 128.69, 144.65, 168.60,170.32, 172.15, 174.80. HRMS calc. for C₃₅H₄₅N₄O₅: 601.3390; found: 601.3392.

### Example 5: Oligonucleotide Synthesis

All DNA oligonucleotides were chemically synthesized on the Applied Biosystems Inc. (ABI) 394 DNA/RNA synthesizer using standard phosphoramidite chemistry. A solution of phosphoramidite 1 in anhydrous DCM (0.12 M) or 2 in anhydrous ACN (0.12M) was used for the coupling, with an extended coupling time of 10 min. Prepacked 1 μmol functionalized controlled pore glass column (Glen Research) was used for 5′ or internal tagged oligonucleotide and 1 μmol UnyLinker (Chemgenes) was used for 3′ tagged oligonucleotide. Oligonucleotides were cleaved from the support and deprotected with concentrated aq. NH₃ at 55° C. for 16 h. The oligonucleotide was then purified by reverse phase HPLC (250×10 mm, Waters, XBridge BEH C18) with a gradient 5% to 35% ACN over 8 min followed by 35% to 100% ACN in 12 min using ACN/TEAA mobile phase mixture. DMT was removed using 2% or 3% TFA solution, for PO or PS oligonucleotide respectively, on a PolyPak cartridge (Glen Research). The purified oligonucleotide was then desalted using Glen Pak 2.5 desalting column (Glen Research). The desalted oligonucleotide was then lyophilized, dissolved in deionized water and quantified by UV absorbance at 260 nm.

### Example 6: Peptides Preparation

All peptides were purchased from Chempeptide Limited. The peptide was dissolved in DI water to make up a stock solution of 5 mM for Pep1 and Pep3, 9.95 mM for Pep2 and 0.72 mM for Pep4.

### Example 7: Synthesis of Adapter Ac-E(NGL)LPETGGW-NH₂ (SEO ID NO:7)

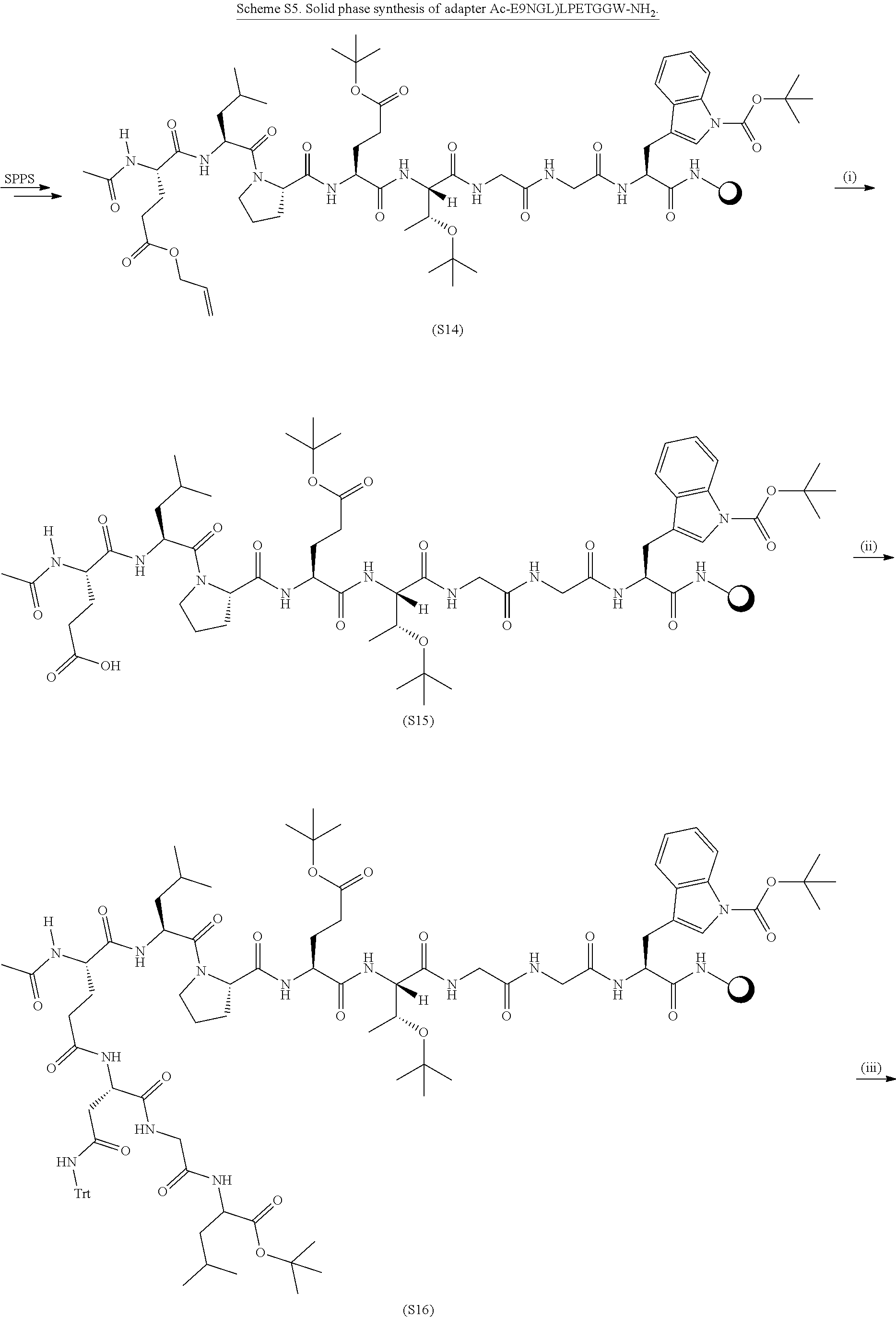

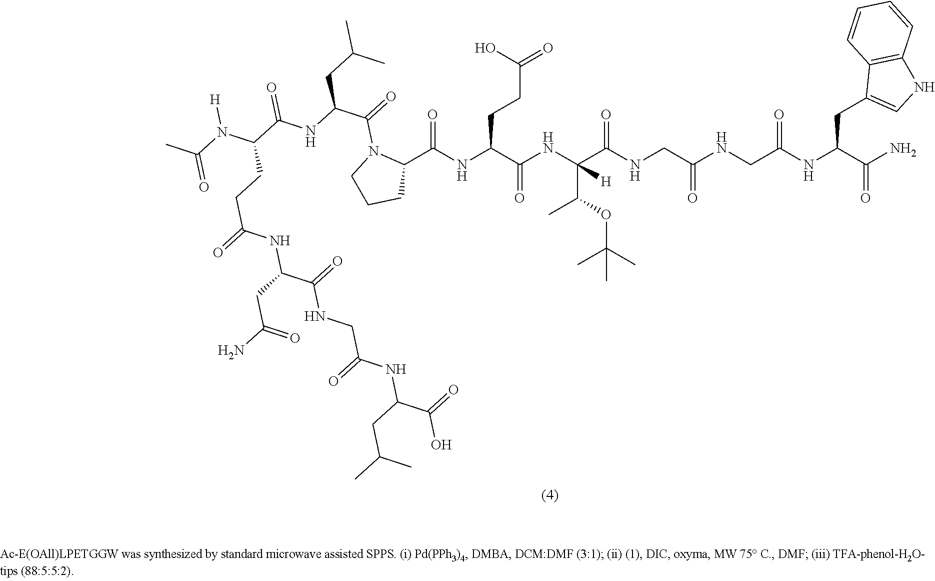 [SMILES: CC(=O)N[C@@H](CCC(=O)N[C@@H](CC(N)=O)C(=O)NCC(=O)NC(CC(C)C)C(=O)O)C(=O)N[C@@H](CC(C)C)C(=O)N1CCC[C@H]1C(=O)N[C@@H](CCC(=O)O)C(=O)N[C@H](C(=O)NCC(=O)NCC(=O)N[C@@H](Cc1c[nH]c2ccccc12)C(N)=O)[C@@H](C)OC(C)(C)C ; "(4)"]

The peptide S14, Ac-E(OAII)LPETGGW (SEQ ID NO:8), was synthesized using a Biotage Initiator+Alstra microwave peptide synthesizer automatically on Rink Amide resin (GL-biochem, 0.65 mmol/g) on a 0.2 mmol scale in a 10 mL reactor vail using Fmoc chemistry. Resin was swelled for 20 min at 70° C. Fmoc deprotection was performed at room temperature in two stages, first by treating the resin with 20% piperidine in DMF for 3 min followed by 20% piperidine in DMF for 10 min. Coupling was performed at 75° C. for 5 mins using 5 eq. of Fmoc-amino acid monomer, 5 eq. Oxyma and 5 eq. DIC in DMF. The N-terminal was capped using 5 eq. of acetic anhydride, 6 eq. of pyridine at room temperature for 20 min. OAll protecting group was removed under nitrogen atmosphere using Pd(PPh₃)₄ (24 mg), DMBA (310 mg), dissolved in DCM:DMF (3:1, 4 mL) for 2 h. The deprotection cocktail was drained and the resin was washed with 0.8 M LiCl in DMF to give the resin bound S15. The tripeptide 3 was double coupled on to S15 at 75° C. for 5 min using 5 eq. of 3, 5 eq. Oxyma and 5 eq. DIC for each coupling. The resin was then washed with DCM after the synthesis was completed and dried under vacuum. The peptide was cleaved from the solid support using a cocktail of TFA-phenol-H₂O-TIPS (88:5:5:2) at room temperature for 3.5 h. The cleavage cocktail was evaporated by nitrogen flow, the crude product precipitated from cold ether and dried under vacuum. The product was then purified by HPLC using a reversed-phase preparative column (10 mm∴250 mm, XBridge Peptide BEH C18 OBD) with a linear gradient 10% to 70% ACN over 25 min using ACN/H₂O mobile phase mixture containing 0.1% TFA. Solvent was removed by lyophilization to give the adapter peptide 4. The product was then reconstituted in deionized water and quantified by UV absorbance at 280 nm. MALDI-TOF MS calc.: 1212.6; found [M+3Na]⁺: 1280.9.

### Example 8: Sortase A Expression

The plasmid containing gene fragment encoding the Sortase A mutant, Sortase-7M (SEQ ID NO:19 with a C-terminal 6×His tag), was purchased from Addgene and transformed into Rosetta T1 R cells. The cells were grown in LB containing antibiotics to OD₆₀₀ 0.6-0.8, and induced with addition of 0.5 mM IPTG at 18° C. overnight. Cells were harvested by centrifugation and the cell pellet resuspended in lysis buffer containing 50 mM Tris-HCl, pH 7.5, 150 mM NaCl, 10% (v/v) glycerol. Cells were lysed by sonication, and the crude lysate clarified by centrifugation at 21,000 rpm at 4° C. for 1 hour. The soluble protein was purified by Ni-affinity chromatography (HisTrap, GE Healthcare) followed by size exclusion chromatography (GE Healthcare) pre-equilibrated with 50 mM Tris-HCl, pH 7.5, 150 mM NaCl, 10% (v/v) glycerol. The sortase-7M protein was concentrated using 10 kDa MWCO centricon (Millipore), concentration determined using bicinchoninic acid protein assay (Pierce) and stored at −80° C.

### Example 9: OaAEP1b Expression

A gene fragment encoding for an N-terminal histidine-tag with human ubiquitin (1-76 aa), and OaAEP1b (24-474 aa) with C247A mutation (SEQ ID NO:20) was synthesized (IDT), cloned into pET-28b vector (Novagen) and transformed into Rosetta T1R cells. The cells were expressed and purified as described by Yang and coworkers (Yang et al. J. Am. Chem. Soc. 2017, 139 (15), 5351-5358) with slight modification to the procedures. Briefly, cells were grown in LB containing antibiotics to OD₆₀₀ 0.6-0.8, and induced with addition of 0.5 mM IPTG, shaking at 220 rpm at 16° C. overnight. Cells were harvested by centrifugation at 10,000 rpm at 4° C. for 10 mins and the cell pellet resuspended in lysis buffer containing 50 mM Tris-HCl pH 7.5, 150 mM NaCl, 10% (v/v) glycerol and 0.05% (v/v) Nonidet P-40. Cells were lysed by sonication, and the crude lysate clarified by centrifugation at 21,000 rpm at 4° C. for 20 min. The soluble protein was purified by Ni-affinity chromatography (HisTrap, GE Healthcare) and anion exchange chromatography (HiTrapQ, GE Healthcare). The purified protein fractions were combined in buffer containing 20 mM HEPES pH 7.0, 2 mM DTT, 10% (v/v) glycerol. The OaAEP1b protein was activated by the removal of the cap domain of OaAEP1 b with the addition of glacial acetic acid to achieve pH 4.0 in the sample. The protein was left at room temperature overnight, and the completeness of the reaction monitored by SDS-PAGE. The activated OaAEP1b protein was concentrated using 10 kDa MWCO centricon (Millipore) and the protein concentration determined using bicinchoninic acid protein assay (Pierce), and stored at −80° C.

### Example 10: Cloning and Expression of Cyan Fluorescent Protein (CFP)

Cyan fluorescent protein (CFP_{PAL})
(SEQ ID NO: 9)
MGSSHHHHHHSQDPMVSKGEELFTGVVPILVELDGDVNGHKFSVSGEGEG
DATYGKLTLKFICTTGKLPVPWPTLVTTLTWGVQCFSRYPDHMKQHDFFK
SAMPEGYVQERTIFFKDDGNYKTRAEVKFEGDTLVNRIELKGIDFKEDGN
ILGHKLEYNYISHNVYITADKQKNGIKANFKIRHNIEDGSVQLADHYQQN
TPIGDGPVLLPDNHYLSTQSKLSKDPNEKRDHMVLLEFVTAAGITLGMDE
LYKNGL

A gene fragment encoding CFPPAL was cloned into pETDuet-1 vector (Novagen) and transformed into Rosetta T1 R cells. The cells were grown in LB containing antibiotics to OD₆₀₀0.6-0.8, and induced with addition of 0.5 mM IPTG, shaking at 220 rpm at 18° C. overnight. Cells were harvested by centrifugation at 10,000 rpm at 4° C. for 7 mins and the cell pellet resuspended in lysis buffer containing 20 mM Kpi pH 7.0, 500 mM KCl, 2 mM DTT, 10% (w/v) glycerol, 20 mM imidazole. Cells were lysed by sonication, and the crude lysate clarified by centrifugation at 21,000 rpm at 4° C. for 1 hour. The crude lysate was filtered before loaded into the Ni-NTA column (Qiagen) for affinity purification. The purified protein fractions were combined and concentrated in buffer containing 20 mM Kpi pH 7.0, 150 mM KCl. using 10 kDa MWCO centricon (Millipore).

Cyan fluorescent protein (CFP_{SORT})
(SEQ ID NO: 10)
MVSKGEELFTGVVPILVELDGDVNGHKFSVSGEGEGDATYGKLTLKFICT
TGKLPVPWPTLVTTLTWGVQCFSRYPDHMKQHDFFKSAMPEGYVQERTIF
FKDDGNYKTRAEVKFEGDTLVNRIELKGIDFKEDGNILGHKLEYNYISHN
VYITADKQKNGIKANFKIRHNIEDGSVQLADHYQQNTPIGDGPVLLPDNH
YLSTQSKLSKDPNEKRDHMVLLEFVTAAGITLGMDELYKLPETGLEHHHH
HH

A gene fragment encoding CFP_{SORT} was cloned into pET-28b vector (Novagen) and transformed into Rosetta T1R cells. The cells were grown in LB containing antibiotics to OD₆₀₀0.6-0.8, and induced with addition of 0.5 mM IPTG, shaking at 220 rpm at 18° C. overnight. Cells were harvested by centrifugation at 10,000 rpm at 4° C. for 7 mins and the cell pellet resuspended in lysis buffer containing 20 mM Kpi pH 7.0, 500 mM KCl, 2 mM DTT, 10% (v/v) glycerol, 20 mM imidazole. Cells were lysed by sonication, and the crude lysate clarified by centrifugation at 21,000 rpm at 4° C. for 1 hour. The crude lysate was filtered before loaded into the Ni-NTA column (Qiagen) for affinity purification. The purified protein fractions were combined and concentrated in buffer containing 50 mM Tris HCl, pH 7.5, 150 mM NaCl, using 10 kDa MWCO centricon (Millipore).

### Example 11: Ligation Reaction with Peptides

The ligation reactions using OaAEP1b were performed in 20 μL reaction mixtures containing 20 mM phosphate buffer, OaAEP1b ligase (0.01 to 0.3 eq.), peptide substrate (200 μM) and oligonucleotide substrate (500 uM to 1 mM) at 37° C. unless otherwise stated.

The ligation reactions using Sortase A were performed in 20 μL reaction mixtures containing 50 mM Tris HCl, 150 mM NaCl (pH 7.5) buffer, Sortase A ligase (1 to 3 eq), peptide substrate (200 μM) and oligonucleotide substrate (1 mM) at 4° C. unless otherwise stated.

The conjugated chimeric product was purified using a reversed-phase analytical column (250×4.6 mm, YMC-Triart C18) with a linear gradient from 5% to 40% acetonitrile/TEAA over 35 min on a Nexera UHPLC system (Shimadzu). A linear fit of ODNref peak area at 260 nm against concentration (10, 50, 100, 150 and 200 μM) was used as the calibration plot. The peak at 260 nm corresponding to the conjugated chimeric product was integrated and the yield was then derived from the linear calibration plot. The identity of the HPLC peaks were verified by MALDI-TOF MS (JEOL JMS-S3000) analysis.

### Example 12: Ligation Reaction with CFP

The ligation reactions using OaAEP1b were performed in 5 μL reaction mixtures containing 20 mM phosphate buffer pH 7.4, protein substrate (100 μM), oligonucleotide substrate (1 mM) and OaAEP1b ligase (0.02 eq) at 37° C. for 1 h. The reaction was monitored by SDS-PAGE.

The ligation reactions using Sortase A were performed in 5 μL reaction mixtures containing 50 mM Tris HCl, 150 mM NaCl (pH 7.5) buffer, protein substrate (50 μM), oligonucleotide substrate (500 μM) and Sortase A ligase (1 eq) at 4° C. for 16 h. The reaction was monitored by SDS-PAGE.

TABLE 1
Oligonucleotide sequences used in this study.

| Oligo^{[a]} | Sequence (5′ → 3′)^{[b],[c],[d],[e]} | Remarks |
|---|---|---|
| ODNref | CTATTTGGATGTCAGC (SEQ ID NO: | Native |
| | 11) | |
| ODN1 | /Tag_{SORT}/CTATTTGGATGTCAGC | 5′-Gly-Gly-Gly tagged |
| | (SEQ ID NO: 11) | |
| ODN2 | /Tag_{SORT}/*C*T*A*T*T*T*G*G*A*T* | 5′-Gly-Gly-Gly tagged PS |
| | G*T*C*A*G*C (SEQ ID NO: 11) | backbone |
| ODN3 | CTATTTGGATGTCAGC/Tag_{SORT}/ | 3′-Gly-Gly-Gly tagged |
| | (SEQ ID NO: 11) | |
| ODN4 | CTATTTGG/Tag_{SORT}/ATGTCAGC | Internal Gly-Gly-Gly tagged |
| ODN S1 | /Tag_{SORT}/TTTTTTTTTT (SEQ ID | 5′-Gly-Gly-Gly tagged poly dT |
| | NO: 12) | |
| ODN S2 | /Tag_{SORT}/GCATTCTAATAGCAGC | 5′-Gly-Gly-Gly tagged |
| | (SEQ ID NO: 13) | |
| ODN S3 | /Tag_{SORT}/TTGGGTGGGTGGGTGGGT | 5′-Gly-Gly-Gly tagged G4 |
| | (SEQ ID NO: 14) | |
| ODN S4 | TTGGGTGGG/Tag_{SORT}/GGGTGGGT | Internal loop Gly-Gly-Gly |
| | | tagged G4 |
| ODN5 | /Tag_{PAL}/CTATTTGGATGTCAGC | 5′-Gly-Leu tagged |
| | (SEQ ID NO: 11) | |
| ODN6 | /Tag_{PAL}/C*T*A*T*T*T*G*G*A*T*G* | 5′-Gly-Leu tagged PS backbone |
| | T*C*A*G*C (SEQ ID NO: 11) | |
| ODN7 | /Tag_{PAL}/C*T*A*T*T*T*G*G*A*T*G* | 5′-Gly-Leu tagged PS LNA |
| | T*C*A*G*C (SEQ ID NO: 11) | gapmer |
| ODN8 | CTATTTGGATGTCAGC/Tag_{PAL}/ | 3′-Gly-Leu tagged |
| | (SEQ ID NO: 11) | |
| ODN9 | CTATTTGG/Tag_{PAL}/ATGTCAGC | Internal Gly-Leu tagged |
| ODN S5 | /Tag_{PAL}/*C*T*A*T*T*T*G*G*A*T* | 5′-Gly-Leu tagged PS backbone |
| | G*T*C*A*G*C (SEQ ID NO: 11) | |
| ODN S6 | /Tag_{PAL}/TTTTTTTTTT (SEQ ID NO: | 5′-Gly-Leu tagged poly dT |
| | 12) | |
| ODN S7 | GCATTCTAATAGCAGC/Tag_{PAL}/ | 3′-Gly-Leu tagged |
| | (SEQ ID NO: 13) | |
| ODN S8 | /Tag_{PAL}/SS/CTATTTGGATGTCAGC | 5′-Gly-Leu tagged disulfide |
| | (SEQ ID NO: 11) | containing oligo |
| ODN S9 | TTGGGTGGG/Tag_{PAL}/GGGTGGGT | Internal loop Gly-Leu tagged |
| | (SEQ ID NO: 14) | G4 |

^{[a]}ODN1-4 and ODN S1-S4 are labeled with (Gly-Gly-Gly) tag, while ODN5-8 and ODN S5-S9 are labeled with (Gly-Leu) tag.
^{[b]}Ligation tags for sortase A (Gly-Gly-Gly) and OaAEP1b (Gly-Leu) are represented as /Tag_{SORT}/ and /Tag_{PAL}/, respectively.
^{[c]}Phosphorothioate-modified inter-nucleotide linkages are marked with asterisks (*).
^{[d]}/SS/ represents C₆-disulfide-C₆ modifier (https://www.glenresearch.com/10-1936.html).
^{[e]}LNA nucleotides are underlined.

TABLE 2
Peptide Sequences Used in This Study.

| Peptide | Sequence (N → C)^{[a]} |
|---|---|
| Pep1 | KALVILPETGLE (SEQ ID NO: 15) |
| Pep2 | H(A_{ib})EGTFTSDVSSYLEGQAAKEFIAWL |
| | VKGRGLPETGLE (SEQ ID NO: 16) |
| Pep3 | KALVINGL (SEQ ID NO: 17) |
| Pep4 | H(A_{ib})EGTFTSDVSSYLEGQAAKEFIAWL |
| | VKGRGNGL (SEQ ID NO: 18) |

^{[a]}Cognate ligase sequence motifs of the ligation handles are underlined.
[b] 2-aminoisobutyric acid substitution is represented as (A_{ib}).

TABLE 3
Ligation reaction conditions and POC characterization by
MALDI-TOF mass spectrometry. Unless otherwise specified,
all reactions were performed in 20 μL reaction mixture
with peptide concentration of 200 μM and 5 eq. (for sortase)
or 2.5 eq. (for OaAEP1b) of oligonucleotide. Yield was estimated
based on area of HPLC peak monitored at 260 nm.

| | | | | | Mass | Estimated |
|---|---|---|---|---|---|---|
| Oligo | Peptide | Cond.^{[a]} | Ligase | | (exp./det.) | Yield |
| ODNref | Pep1 | I | 1 | eq. | N.A. | N.A. |
| ODN1 | Pep1 | I | — | | N.A. | N.A. |
| ODN1 | Pep1 | I | 1 | eq. | 6190.6/6190.1 | 79% |
| ODN2 | Pep1 | I | 1 | eq. | 6447.5/6447.8 | 100% |
| ODN3 | Pep1 | I | 1 | eq. | 6190.6/6192.1 | 14% |
| ODN4 | Pep1 | I | 1 | eq. | 6190.6/6190.7 | 49% |
| ODN2 | Pep2 | II | 3 | eq. | 9274.5/9276.9 | 23% |
| ODNref | Pep3 | III | 0.01 | eq. | N.A. | N.A. |
| ODN5 | Pep3 | III | — | | N.A. | N.A. |
| ODN5 | Pep3 | III | 0.01 | eq. | 5875.3/5874.2 | 93% |
| ODN6 | Pep3 | III | 0.01 | eq. | 6116.2/6113.0 | 52% |
| ODN7 | Pep3 | IV | 0.1 | eq. | 6310.2/6311.3 | 100% |
| ODN8 | Pep3 | III | 0.01 | eq. | 5875.3/5873.8 | 94% |
| ODN9 | Pep3 | III | 0.01 | eq. | 5875.3/5873.4 | 52% |
| ODN5 | Pep4 | V | 0.1 | eq. | 8702.3/8701.9 | 43% |
| ODN6 | Pep4 | VI | 0.3 | eq. | 8944.2/8947.3 | 17% |

^{[a]}Condition I: 50 mM Tris HCl (pH 7.5), 150 mM NaCl, 4° C., 16 h, 1 eq. Sort-7M. Condition II: 50 mM Tris HCl (pH 7.5), 150 mM NaCl, 4° C., 16 h, 3 eq. Sort-7M. Condition III: 20 mM KPi (pH 7), 37° C., 30 min; Condition IV: 20 mM KPi (pH 7), 37° C., 1.5 h, 0.1 eq. OaAEP1b; Condition V: 20 mM KPi (pH 7), 37° C., 16 h, 0.1 eq. OaAEP1b; Condition VI: 20 mM KPi (pH 9), 37° C., 16 h, 0.3 eq OaAEP1b, peptide-to-oligonucleotide ratio of 1:5.

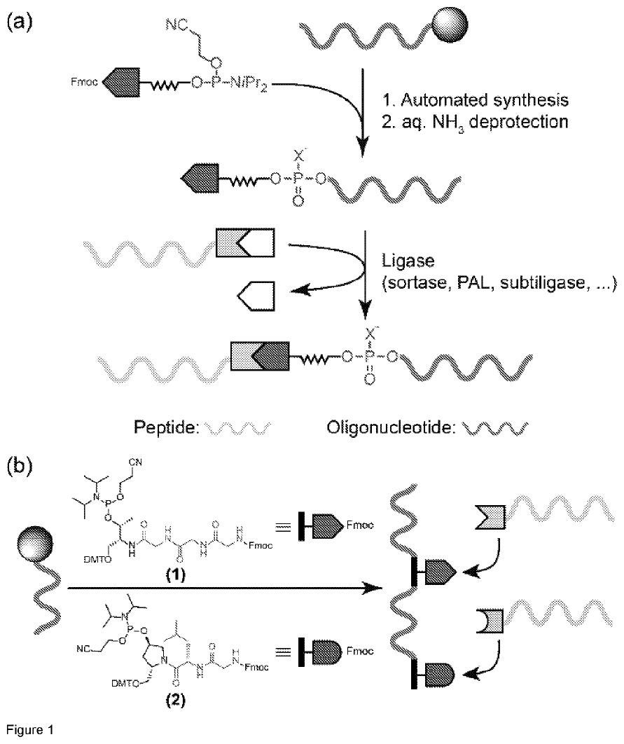

Herein disclosed is a phosphoramidite tag-based approach to enzymatic ligation of peptides and oligonucleotides that greatly simplifies the preparation of POCs (FIG. 1). A short peptide recognition motif (≤3 amino acids) was converted to a tag phosphoramidite that can be readily incorporated onto an oligo during automated synthesis (FIG. 1a, top), followed by ligation with the peptide counterpart containing the matching ligation handle by the cognate ligase (FIG. 1a, bottom). The utility of this method was demonstrated with sortase (Mazmanian et al. Science 1999, 285 (5428), 760-3; Mao et al. J. Am. Chem. Soc. 2004, 126 (9), 2670-1; Chen et al. Proc Natl Acad Sci USA 2011, 108 (28), 11399-404; Popp & Ploegh, Angew. Chem. Int. Ed. Engl. 2011, 50 (22), 5024-32) and peptide asparaginyl ligase (PAL) (Nguyen et al. Nat. Chem. Biol. 2014, 10 (9), 732-8; Harris et al. Nat Commun 2015, 6, 10199; Yang et al. J. Am. Chem. Soc. 2017, 139 (15), 5351-5358; Jackson et al. Nat Commun 2018, 9 (1), 2411; Hemu et al. Proc Nati Acad Sci USA 2019, 116 (24), 11737-11746), two distinct classes of ligase that have seen widespread use in protein engineering. The ligation strategy was successfully applied across a diverse range of oligo and peptide constructs, and was further extended to the ligation of a protein with oligo. The modular nature of tag incorporation further provides opportunity for the conjugation of multiple peptides/proteins onto an oligo in a controllable manner. Thus, our approach provides a straightforward path towards the streamlined development and production of POCs.

Sortase is a family of transpeptidases that anchor proteins onto bacterial cell wall. Sortase A from Staphylococcus aureus ligates N-terminal poly-glycine onto a C-terminal end containing the consensus sorting motif LPXTG. Accordingly, a phosphoramidite tag (1) containing the Gly-Gly-Gly ligation motif (FIG. 1b, red), starting from a threoninol (S1) scaffold was synthesized (Scheme S1). Compound 1 is fully compatible with phosphoramidite-based solid-phase oligonucleotide synthesis, hence allowing straightforward incorporation of the Gly-Gly-Gly tag onto oligonucleotides (FIG. 1a, top).

It was proceeded with ligation of a 5′-(Gly-Gly-Gly)-tagged 16-nucleotide (nt) oligo sequence (Hong et al., Sci. TransL Med. 2015, 7(314), 314ra185) (ODN1, Table 1) against a model peptide (Pep1, Table 2) containing the cognate ligation handle using recombinant sortase A (VVuethrich et al. PLoS One 2014, 9 (10), e109883; Witte et al. Nature Protocols 2015, 10, 508). The ligation reaction was performed in 50 mM Tris-HCl (pH 7.5) buffer containing 150 mM NaCl at 4° C. for 16 hours, at a peptide-to-oligonucleotide ratio of 1:5. The ligation product was isolated with reversed-phase (RP)-HPLC, and its mass was verified by MALDI-TOF (Table 3). As a negative control, no ligation products were observed when sortase A was excluded from the reaction mixture, nor when the reference oligo (ODNret) with no tag was used for the reaction (Table 3). Ligation of a 5′-(Gly-Gly-Gly)-tagged fully phosphorothioate (PS)-modified oligo of the same sequence (ODN2, Table 1) with Pep1 was similarly successful (Table 3), validating the applicability of the approach towards conjugation with therapeutic oligos, which have seen widespread use of PS backbone chemistry for improved nuclease resistance. In addition, POC ligation was achieved regardless of the alternate placement of the tag either at the 3′-end (ODN3) or at an internal position (ODN4) of the oligo (Table 3). Furthermore, ODN2 was also successfully ligated onto a 38-amino-acid (aa) long glucagon-like peptide 1 (GLP1) variant containing the sorting motif on its C-terminus (Pep2, Table 2; Table 3).

POC ligation using PALwas carried out next. PAL is a family of asparaginyl-specific ligases found in cyclotide-producing plants, among which include butelase 1 (Nguyen et al., supra) and OaAEP1b (Harris et al., supra; Yang et al., supra). Native substrates of OaAEP1b comprise the peptide sequences GL and NGL at the N- and C-terminus, respectively. In this case, we synthesized a phosphoramidite tag (2) containing the (Gly-Leu) ligation motif (FIG. 1b), which is fully compatible with solid-phase oligonucleotide synthesis, starting from a hydroxyprolinol (S4) scaffold (Scheme S2). Native, PS-modified, and locked nucleic acid (LNA) gapmer version of ODNref 5′-tagged with (Gly-Leu) (ODN5, ODN6, and ODN7, respectively, Table 1) were all successfully conjugated onto a model peptide (Pep3, Table 2) containing the cognate ligation handle using recombinant OaAEP1b in 20 mM potassium phosphate buffer (pH 7), at a peptide-to-oligonucleotide ratio of 1:2.5 (Table 3). The ligation products were obtained after only 30 minutes of incubation with OaAEP1b, thanks to high efficiency of the PAL family. Ligation of Pep3 with various other oligo constructs, either consisting of different placement of the ligation tag (ODN8 and ODN9) or comprising additional oligo modification (ODN S8 with a disulfide modifier, Table S1), was similarly successful. On the other hand, ligation of a 34-aa GLP1 variant containing the ligation handle on its C-terminus (Pep4, Table 2) with ODN5 and ODN6 were also successful (Table 3) after incubation with the ligase for 16 hours.

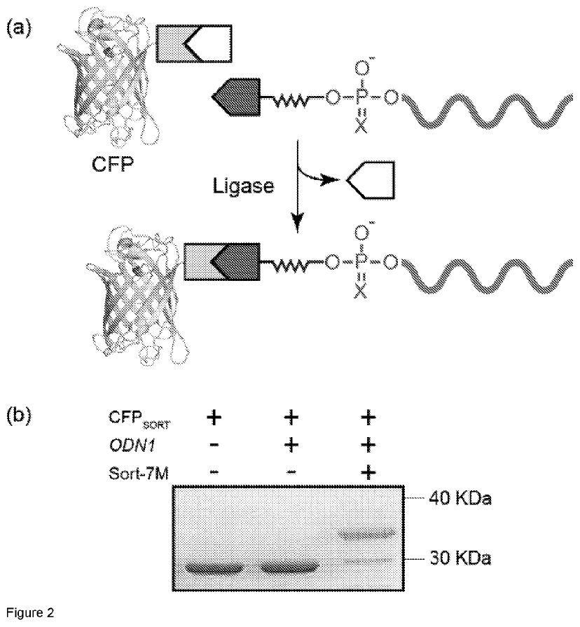

Having demonstrated adaptability of the tag-based approach towards ligation of various peptides and oligonucleotides, next its application towards the ligation of a protein with oligonucleotide was investigated. To that end, a cyan fluorescent protein (CFP) variant was engineered to harbor an LPETG (SEQ ID NO:6) (for sortase A; CFP_{SORT}) or NGL (for OaAEP1b; CFP_{PAL}) ligation handle at the C-terminus (FIG. 2a). Both proteins were ligated against a panel of different oligo constructs with sortase A and OaAEP1b accordingly. For sortase A, successful CFP ligation for all constructs in general was observed (FIG. 2b), with ODN1, ODN2, and a poly-dT oligo (ODN S1, Table S1) giving particularly high yields. For OaAEP1b, also successful CFP ligation for the tested constructs in general was observed (data not shown), even with just 1 hour of incubation time. These examples validated the tag-based approach towards protein-oligonucleotide ligation and should be readily adaptable to the ligation of oligonucleotides with other protein counterparts.

Chemical conjugation has been the prevailing approach towards development of POCs, from design of compatible in-line peptide and oligo synthesis, to functionalization on the peptide and oligo fragments for post-synthetic conjugation. The current invention brings forth an enzymatic dimension to POC generaton. By implementing an in-line incorporaton of ligation motif onto oligo through the design of custom phosphoramidite tags, an oligo can be readily coupled with peptide/protein counterparts containing the matching ligation handle by a cognate ligase. Specifically, the utility of the approach was demonstrated with sortase and PAL, two distinct ligase classes that have seen widespread use in peptide and protein engineering.

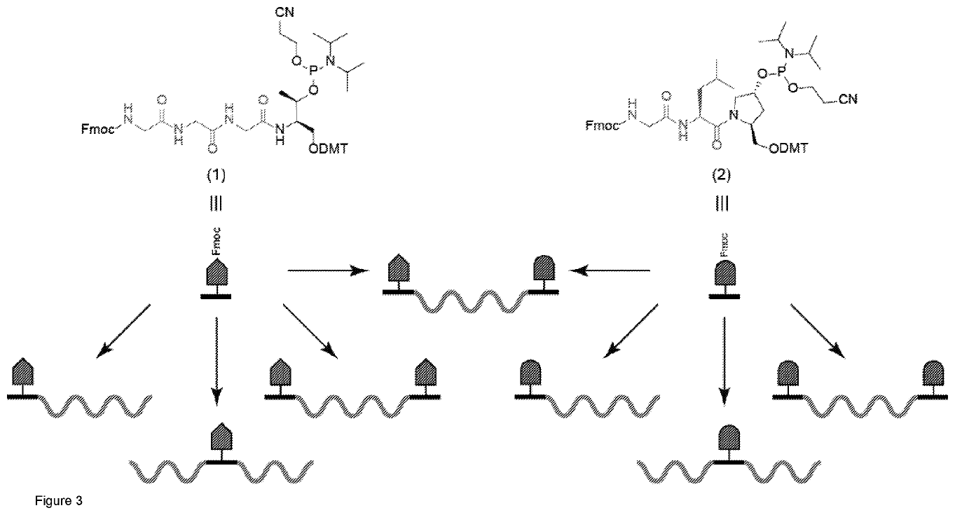

Despite broad adoption of ligases in protein engineering, reports on their adaptation to POC generation were few and far between. Koussa et al. reported on construction of DNA-protein hybrids using sortase (Koussa et al. Methods 2014, 67 (2), 134-41) while Harmand et al. combined sortase A and butelase 1 to generate a C-to-C fusion protein through DNA linkers (Harmand et al. Bioconjug Chem 2018, 29 (10), 3245-3249). In both cases, protein conjugation was localized to the 5′-end of the oligo, and intermediate steps were required in order to prepare the oligo for ligation. In the current invention, the phosphoramidite tag was directly coupled onto the oligo chain during automated solid-phase synthesis and ready for ligation after standard cleavage, deprotection, and purification protocols. Furthermore, it was shown that successful POCs were generated regardless of the point of tag incorporation on the oligo be it at the 5′-/3′-end or at an internal position. In principle, multiple copies of the same phosphoramidite tag can be coupled either in tandem or at discrete loci on an oligo for conjugation with multiple copies of the same peptide/protein (FIG. 3). Different phosphoramidite tags can also be combined to bring about conjugation of two or more distinct peptide(s)/protein(s) onto a single oligo in an addressable manner (FIG. 1b and FIG. 3).

The phosphoramidite tag-based approach was validated against a panel of different oligos, various peptides, and a protein. The diverse nature of the oligo constructs employed (Table S1), ranging from the site of tag attachment (e.g. 5′-end: ODN1/ODN2/ODN5/ODN6/ODN7, 3′-end: ODN3/ODN8; internal: ODN4/ODN9), to the presence of additional modifier (e.g. disulfide modifier: ODN S8) or being fully backbone-modified (e.g. PS: ODN2/ODN6, PS LNA gapmer: ODN7), to the adoption of non-canonical structural forms (e.g. G-quadruplex-forming motif: ODN S3/ODN S4/ODN S9), supported the versatility of the proposed methodology. Furthermore, utility of a ligase for conjugation allowed to leverage the high efficiency and specificity of enzymatic ligation; simple incubation of the peptide and oligo counterparts that contain matching ligation handles with the cognate ligase led to the cleared POCs with minimal side products. In the case of PAL, POCs could be produced in as little as 30 minutes upon incubation with the ligase. No additional activation or preparative steps were needed aside from standard solid-phase synthesis and purification of the peptide and oligo counterparts. With judicious experimental design (e.g. incorporation of Hisx6 label at the peptide/protein C-terminus after the ligation handle, as in CFP_{SORT}), POC purification could be further simplified; the unreacted peptide/protein together with the leaving group and ligase, all of which contain Hisx6 labels, could be readily removed from the reaction mixture by passage through a nickel column, thus allowing straightforward separation of the POC from the excess oligo substrate by liquid chromatography.

In recent years, there have been mounting interests In the conjugation of RNA therapeutics with peptides and proteins to achieve targeted delivery or enhancement in efficacy. The peptide/protein moiety used ranges from short peptide motifs (e.g. integrin-binding RGD) to intermediate-length oligopeptides (e.g. GLP1) or macromolecular antibodies. Herein, it was shown that a GLP1 variant can be ligated with PS oligos at high yields with minimal steps, and the strategy was successfully applied in the context of a bigger protein component. The ligation approach should be readily adaptable to facilitate the development and production of additional peptide-/protein-oligonucleotide conjugates as therapeutics.

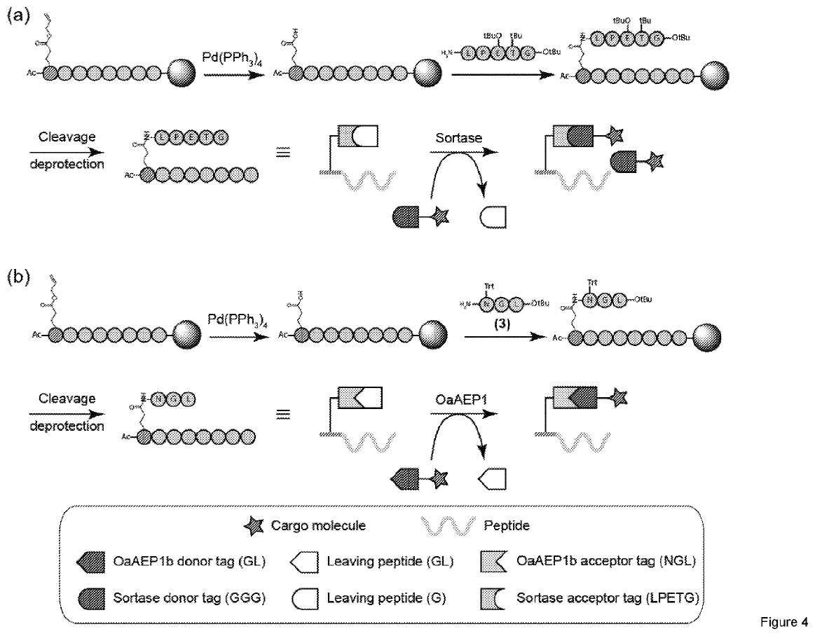

Further, ligation of oligos consisting of disparate configurations with the C-terminus of peptides and proteins was demonstrated. To achieve ligation onto the N-terminus of peptides or proteins, at least two possible alternative strategies can be conceived. The first would involve direct swapping of ligation handles on the peptide and oligo (i.e. LPETG-tag on the oligo, GGG handle on the peptide for sortase A; NOL-tag on the oligo, GL handle on the peptide for OaAEP1b). Accordingly, a LPETG (SEQ ID NO:6) or NGL phosphoramidite tag would lead to incorporation of the ligation handle on the oligo end for subsequent ligation. The second strategy would retain the GGG or GL tag on the oligo end, while projecting out the NGL and LPETG (SEQ ID NO:6) ligation handle from the N-terminus of the peptide. Implementation of the latter strategy on peptides would be relatively straightforward, through coupling of LPETG or NGL (3, Scheme S4) peptide motif onto the N-terminus or side chain of the N-terminal residue (FIG. 4) during solid-phase peptide synthesis. In this manner, the oligo can also be conjugated onto internal residue of peptides through ligation with a matching ligation handle projecting out from side-chain residues. On the other hand, coupling of LPETG or NGL peptide motif on the N-terminus of protein can be achieved through the help of a custom linker or adapter, for instance a bifunctional tag (4, Scheme S5) projecting both LPETG and NGL motifs (FIG. 5). In principle, the use of compounds 3 and 4 are not limited to POC per se, but rather can be applied in a broader scope to bring together a peptide/protein with another entity, e.g. N-to-N fusion of proteins and N-terminal conjugation of protein with small-molecules.

In the design of the two phosphoramidite tags, an aa-like linker (threoninol (S1) for sortase A, hydroxyproline (S4) for OaAEP1b) was chosen on which the tag motif (GGG and GL) was incorporated. These moieties are well-suited as linker due to: (i) a compact scaffold, (ii) full compatibility with peptide synthesis hence sequential coupling of aa residues during tag generaton, and (iii) presence of two hydroxyl groups with different reactivity, of which (iii a) one is converted to amidite functionality for coupling onto oligo and (iii b) the other is attached with a protecting group (e.g. 4,4′-dimethoxytrityl; DMT) to provision for oligo chain extension. Various other scaffolds can potentially be used in place as the linker, for instance a nucleotide phosphoramidite on which the tag motif is labeled directly on any of the sugar, phosphate, or nucleobase constituents, or additionally, any other chemical modification groups on nucleotide analogs, as described herein.

Here Gly-Gly-Gly (sortase A) and Gly-Leu (PAL) were utilized as the ligation motif for the phosphoramidite tag. It was found that the approach should be amenable to variations in the ligation motif, as described herein. For the former, poly-alanine motif was used previously for peptide-protein ligation with sortase A from Streptococcus pyogenes (Raeeszadeh-Sarmazdeh et al. Colloids Surf. B. Biointerfaces 2015, 128, 457-463) In the case of PAL, as has been shown in US patent application 2019/0218586, variants including (Arg-Leu) and (Pro-Leu; S9, Scheme S3) are expected to lead to a similar if not better ligation efficiency with OaAEP1b. Furthermore, members in the family of PAL and AEP display different sequence preferences thus offering additional potential tag motifs for use. The motif selection space can be further expanded considering additional ligase families, for instance subtiligase (Weeks et al. Chem. Rev. 2020, 120 (6), 3127-3160). Regardless of the ligase of choice, the core principle with tag-assisted enzymatic ligation of POC applies.

In conclusion, the use of two separate ligases for POC generation through the development of phosphoramidite tags tailored for each ligase has been successfully demonstrated herein. Diverse oligo and peptide/protein constructs were successfully ligated with high efficiency. The ligation approach provides a straightforward path towards the streamlined development and production of POCs.

All documents referred to herein are incorporated by reference in their entirety.

1. Method for the enzymatic ligation of a (poly)peptide with a cargo molecule, the method comprising the steps of:
(i) providing at least one cargo molecule modified with a peptide tag and at least one poly(peptide) to be ligated to the cargo molecule, wherein the peptide tag and/or the (poly)peptide comprises a ligation motif for a peptide ligase; and
(ii) contacting the cargo molecule and the poly(peptide) with a peptide ligase that ligates the peptide tag with the (poly)peptide via the ligation motif.

2. The method of claim 1, wherein the cargo molecule comprises one or more peptide tags.

3. The method of claim 1, wherein the peptide tag is up to 10 amino acids in length, preferably 2 to 7 amino acids in length.

4. The method of claim 1, wherein the peptide tag is coupled to the cargo molecule via a scaffold moiety.

5. The method of claim 1, wherein the ligation motif for a peptide ligase is located on the C-terminus of the (poly)peptide to be ligated.

6. The method of claim 1, wherein the peptide ligase is selected from sortase and peptidyl asparaginyl ligases (PALs), optionally butelase-1, VyPAL2 or OaAEP1b.

7. The method of claim 1, wherein the cargo molecule is selected from the group consisting of dyes, drugs, aptamers and oligonucleotides.

8. The method of claim 7, wherein the cargo molecule is an oligonucleotide.

9. The method of claim 8, wherein the peptide tag is attached to the 5′ end, the 3′ end or incorporated into the nucleotide chain of the oligonucleotide or any combination thereof.

10. The method of claim 8, wherein the peptide tag is coupled to the backbone, the sugar or the base moieties of the oligonucleotide or any combination thereof.

11. The method of claim 8, wherein the oligonucleotide is an oligonucleotide analogue comprising modified bases, modified sugars, phosphorothioate linkages, phosphorodiamidate morpholino units, locked nucleic acid monomers or combinations thereof

12. The method of claim 8, wherein the oligonucleotide modified with a peptide tag is obtained by reacting the peptide tag conjugated to a scaffold comprising a reactive group that can be coupled to a nucleotide or nucleotide analogue, preferably a phosphoramidite or phosphoramidate group, with the oligonucleotide under conditions that allow coupling of said reactive group with the oligonucleotide to yield the oligonucleotide modified with a peptide tag.

13. The method of claim 12, wherein the scaffold is an amino acid analogue, preferably 4-hydroxyprolinol, serinol, threoninol, N-methyl-serinol, or N-methylthreoninol, each comprising a phosphoramidite or phosphoramidate group, or
(A) where the peptide tag, represented by “Y”, is connected to the scaffold via a carbonyl group, the scaffold is selected from the group consisting of: 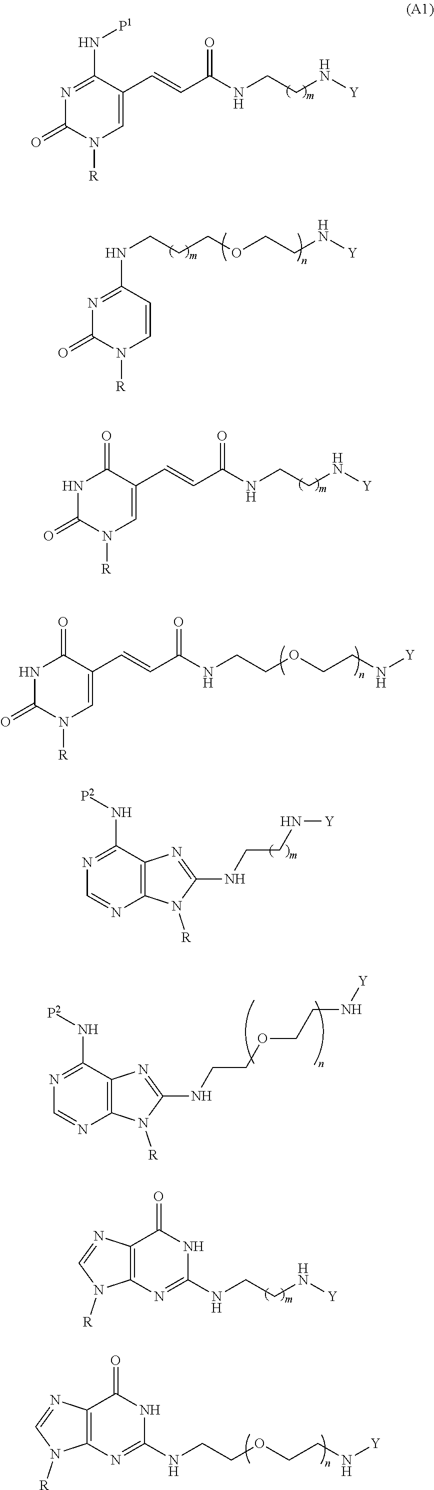 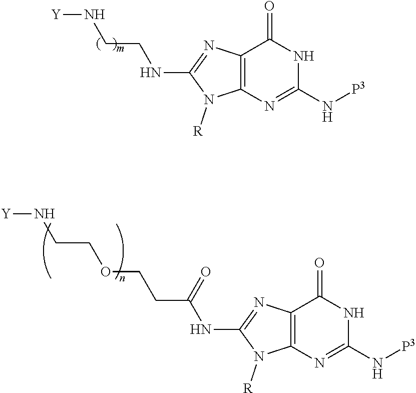
wherein R is selected from 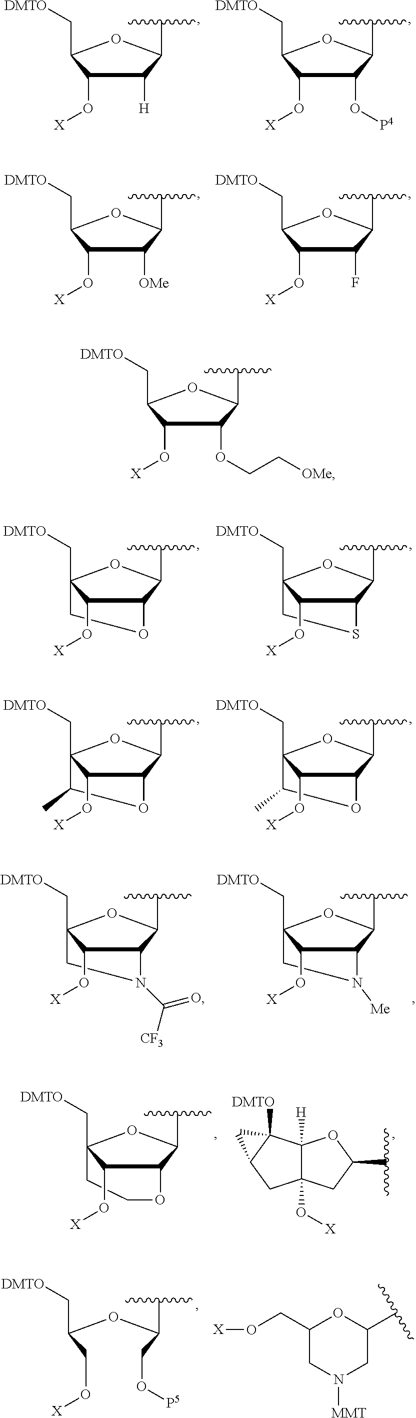
wherein X represents the reactive group and “DMT” represents a protecting group for hydroxyl; or 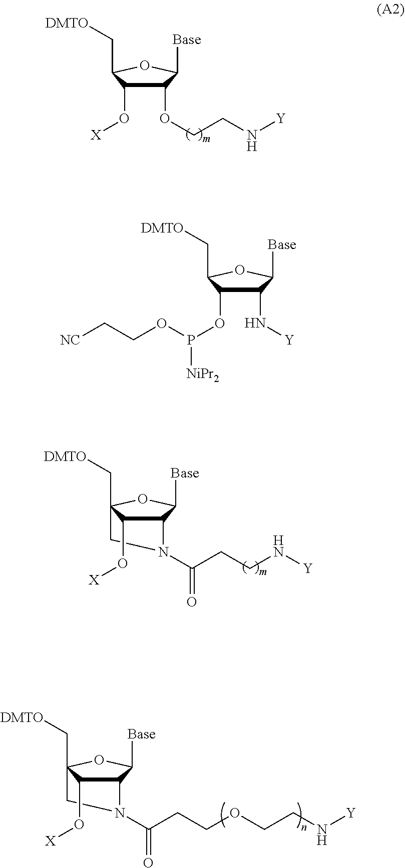
wherein “Base” represents a nucleobase, “DMT” represents a protecting group for hydroxyl and X represents the reactive group; or 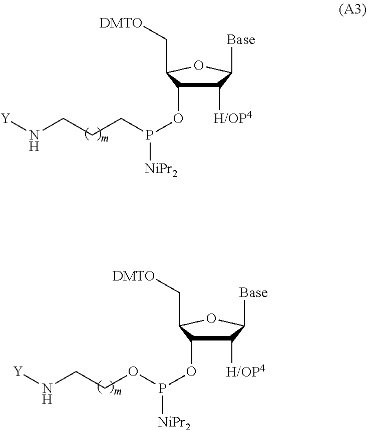
wherein “Base” represents a nucleobase, “DMT” represents a protecting group for hydroxyl and the phosphoramidite group is the reactive group; or 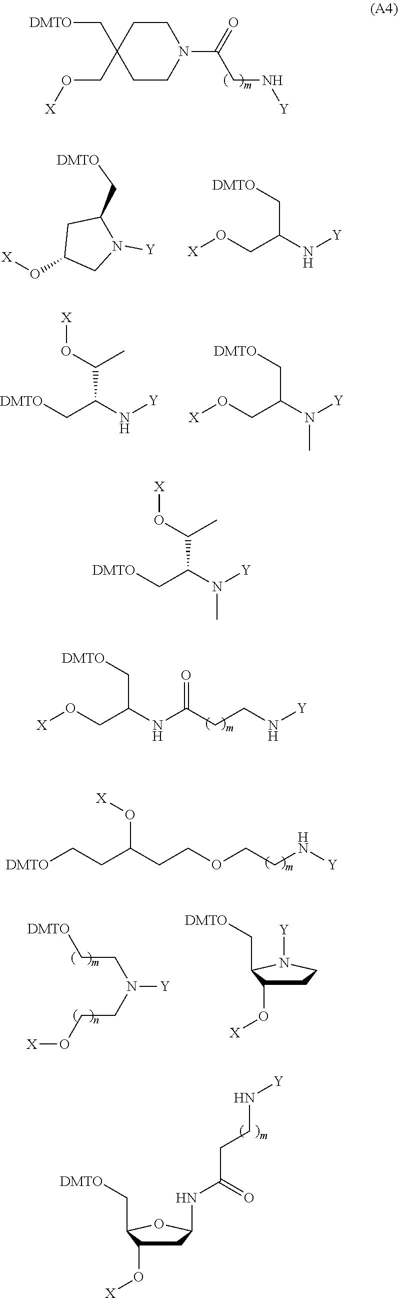
wherein X represents the reactive group and “DMT” represents a protecting group for hydroxyl; or 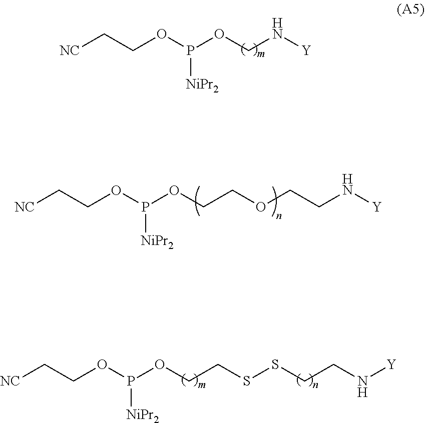
wherein the phosphoramidite group is the reactive group;
wherein in (A1) to (A5) Y is optionally selected from: 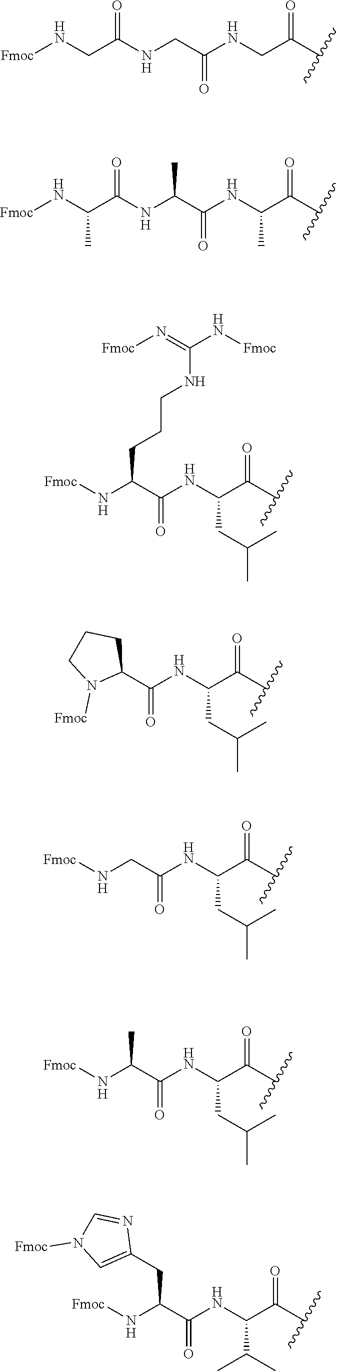
wherein “Fmoc” represents a protecting group for amino and imino groups; or
(B) where the peptide tag, represented by “Z”, is connected to the scaffold via an amino group, the scaffold is selected from the group consisting of: 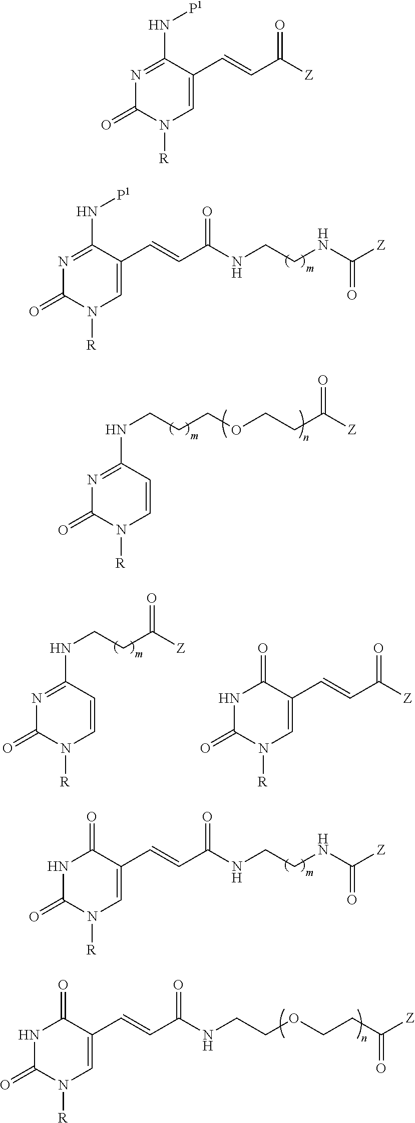 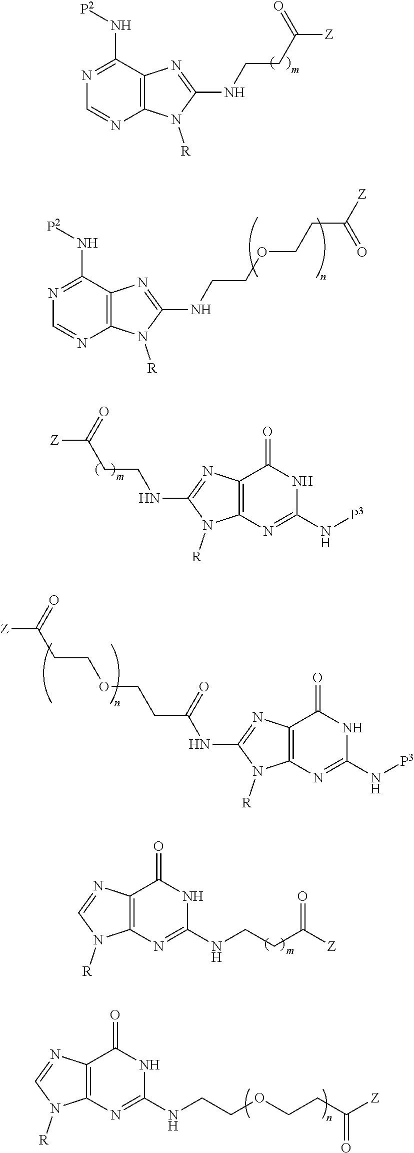
wherein R is selected from 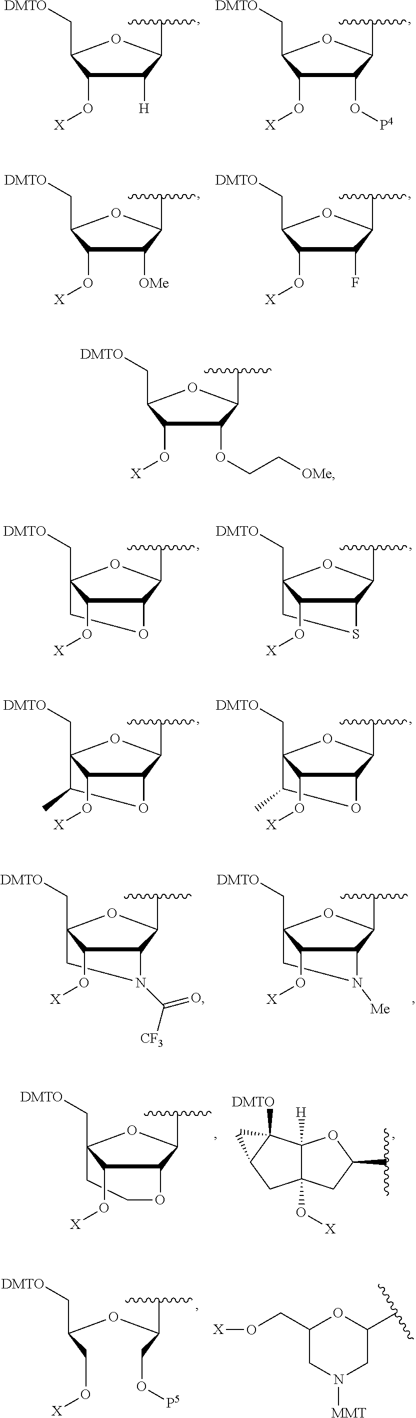
wherein X represents the reactive group and “DMT” represents a protecting group for hydroxyl; or 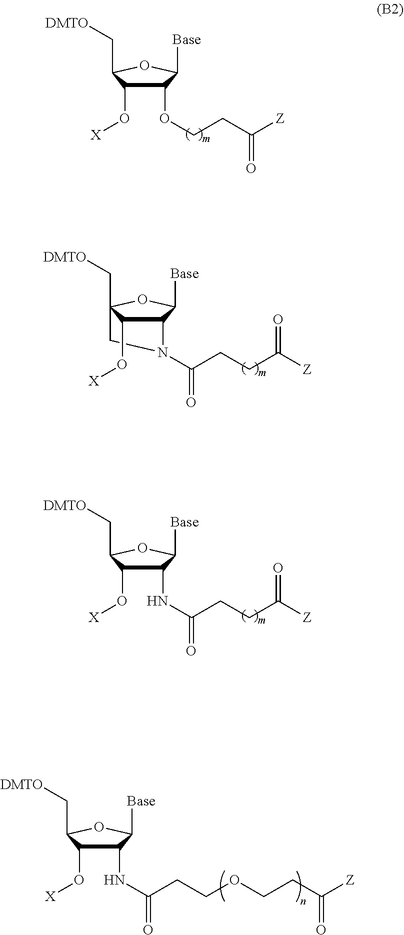
wherein “Base” represents a nucleobase, “DMT” represents a protecting group for hydroxyl and X represents the reactive group; or 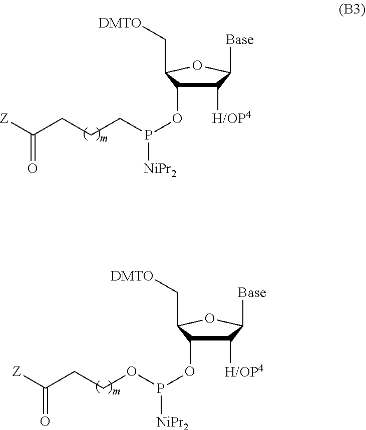
wherein “Base” represents a nucleobase, “DMT” represents a protecting group for hydroxyl and the phosphoramidite group is the reactive group; or 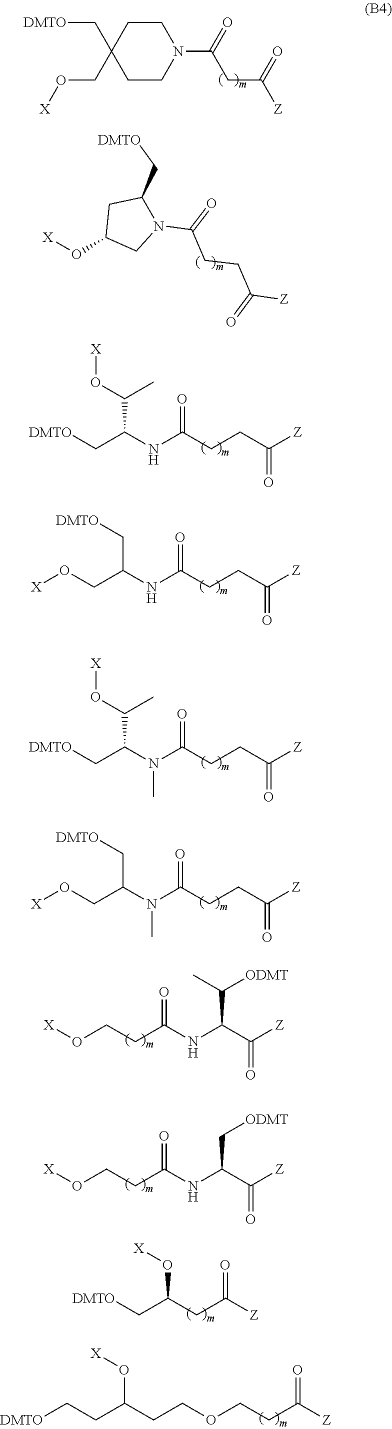 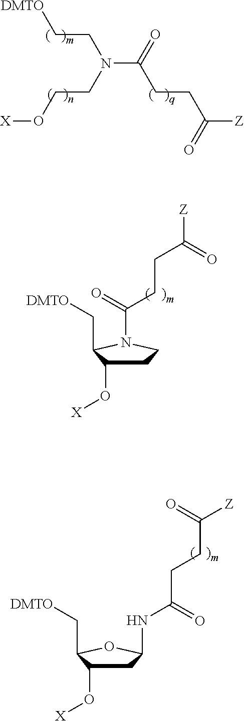
wherein X represents the reactive group and “DMT” represents a protecting group for hydroxyl; or 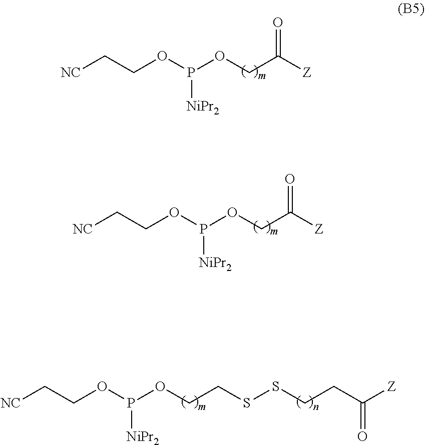
wherein the phosphoramidite group is the reactive group;
wherein in (B1) to (B5) Z is optionally selected from: 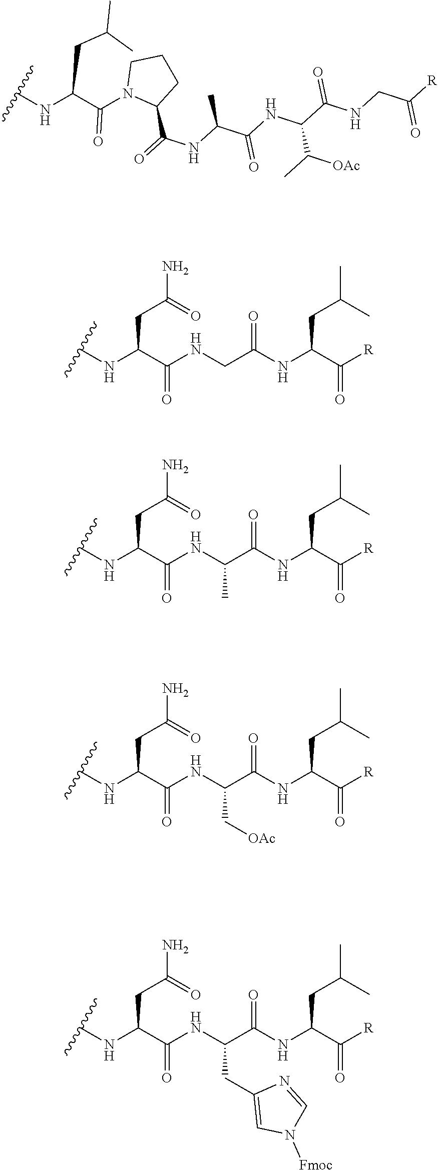
wherein R is NH₂ or O—P⁶, with P⁶ being a carboxylic acid protecting group;
wherein in (A1)-(A5) and (B1)-(B5)
m is 0-8;
n is 0-8;
P¹ is a protecting group on N4 of cytosine suitable for use in SPOS;
P² is a protecting group on N6 of adenine suitable for use in SPOS;
P³ is a protecting group on N2 of guanine suitable for use in SPOS;
P⁴ is a protecting group on 2′-O of pentose sugar suitable for use in SPOS of RNA;
P⁵ is a protecting group suitable for use in SPOS of UNA.

14. The method of claim 1, wherein ligation of the cargo molecule and the poly(peptide) with a peptide ligase is via a bifunctional adapter comprising two ligation motifs for a peptide ligase that can be the same or different.

15. Conjugates obtainable according to the methods of claim 1.


SEQUENCE LISTING (20 sequences)

SEQ ID NO: 1 - protein, 269 aa, Artificial
  VyPAL2 core domain
DSIGTRWAVLIAGSKGYHNYRHQADVCHMYQILRKGGVKDENIIVFMYDDIAYNESNPFPGIIINKPGGENVYKGVPKDYTGEDINNVNFLAAILGNKSAIIGGSGKVLDTSPNDHIFIYYADHGAPGKIGMPSKPYLYADDLVDTLKQKAATGTYKSMVFYVEACNAGSMFEGLLPEGTNIYAMAASNSTEGSWITYCPGTPDFPPEFDVCLGDLWSITFLEDCDAHNLRTETVHQQFELVKKKIAYASTVSQYGDIPISKDSLSVYM

SEQ ID NO: 2 - protein, 281 aa, Artificial
  Butelase-1 active fragment
VEGTRWAVLVAGSKGYVNYRHQADVCHAYQILKKGGLKDENIIVFMYDDIAYNESNPHPGVIINHPYGSDVYKGVPKDYVGEDINPPNFYAVLLANKSALTGTGSGKVLDSGPNDHVFIYYTDHGGAGVLGMPSKPYIAASDLNDVLKKKHASGTYKSIVFYVESCESGSMFDGLLPEDHNIYVMGASDTGESSWVTYCPLQHPSPPPEYDVCVGDLFSVAWLEDCDVHNLQTETFQQQYEVVKNKTIVALIEDGTHVVQYGDVGLSKQTLFVYMGTDPAN

SEQ ID NO: 3 - protein, 474 aa, Oldenlandia affinis
MVRYLAGAVLLLVVLSVAAAVSGARDGDYLHLPSEVSRFFRPQETNDDHGEDSVGTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWCYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDADLLHLWEKFRKAPEGSARKEVAQTQIFKAMSHRVHIDSSIKLIGKLLFGIEKCTEILNAVRPAGQPLVDDWACLRSLVGTFETHCGSLSEYGMRHTRTIANICNAGISEEQMAEAASQACASIP

SEQ ID NO: 4 - protein, 483 aa, Viola yedoensis
MQLFAAGVILFFLLALSGTIAGGLDVDSLQLPSEAAKFFHNDNSTNDDDSIGTRWAVLIAGSKGYHNYRHQADVCHMYQILRKGGVKDENIIVFMYDDIAYNESNPFPGIIINKPGGENVYKGVPKDYTGEDINNVNFLAAILGNKSAIIGGSGKVLDTSPNDHIFIYYADHGAPGKIGMPSKPYLYADDLVDTLKQKAATGTYKSMVFYVEACNAGSMFEGLLPEGTNIYAMAASNSTEGSWITYCPGTPDFPPEFDVCLGDLWSITFLEDCDAHNLRTETVHQQFELVKKKIAYASTVSQYGDIPISKDSLSVYMGTDPANDNRTFVDENSLRPPLKVIHQHDADLYHIWCKYNMAPEGSSKKIEAQKQLLELMSHRAHVDNSITLIGKLLFGVNKASKVLNTVRPVGQPLVDDWQCLKAMIRTFETHCGSLSEYGMKHTLSFANMCNAGIQKEQLAEAAAQACVTFPSNPYSSLAEGFSA

SEQ ID NO: 5 - protein, 482 aa, Clitoria ternatea
MKNPLAILFLIATVVAVVSGIRDDFLRLPSQASKFFQADDNVEGTRWAVLVAGSKGYVNYRHQADVCHAYQILKKGGLKDENIIVFMYDDIAYNESNPHPGVIINHPYGSDVYKGVPKDYVGEDINPPNFYAVLLANKSALTGTGSGKVLDSGPNDHVFIYYTDHGGAGVLGMPSKPYIAASDLNDVLKKKHASGTYKSIVFYVESCESGSMFDGLLPEDHNIYVMGASDTGESSWVTYCPLQHPSPPPEYDVCVGDLFSVAWLEDCDVHNLQTETFQQQYEVVKNKTIVALIEDGTHVVQYGDVGLSKQTLFVYMGTDPANDNNTFTDKNSLGTPRKAVSQRDADLIHYWEKYRRAPEGSSRKAEAKKQLREVMAHRMHIDNSVKHIGKLLFGIEKGHKMLNNVRPAGLPVVDDWDCFKTLIRTFETHCGSLSEYGMKHMRSFANLCNAGIRKEQMAEASAQACVSIPDNPWSSLHAGFSV

SEQ ID NO: 6 - protein, 5 aa, Artificial
  Sortase ligation motif
LPETG

SEQ ID NO: 7 - protein, 8 aa, Artificial
  adapter sequence
  MISC_FEATURE (1)..(1): N-terminus acetylated; side chain linked to peptide NGL
  MISC_FEATURE (8)..(8): C-terminus amidated
ELPETGGW

SEQ ID NO: 8 - protein, 8 aa, Artificial
  modified peptide S14
  MISC_FEATURE (1)..(1): N-terminus acetylated; side chain coupled to allyl
ELPETGGW

SEQ ID NO: 9 - protein, 256 aa, Artificial
  CFP PAL
MGSSHHHHHHSQDPMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSKLSKDPNEKRDHMVLLEFVTAAGITLGMDELYKNGL

SEQ ID NO: 10 - protein, 252 aa, Artificial
  CFP SORT
MVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSKLSKDPNEKRDHMVLLEFVTAAGITLGMDELYKLPETGLEHHHHHH

SEQ ID NO: 11 - DNA, 16 bp, Artificial
  oligonucleotide
ctatttggatgtcagc

SEQ ID NO: 12 - DNA, 10 bp, Artificial
  oligonucleotide
tttttttttt

SEQ ID NO: 13 - DNA, 16 bp, Artificial
  oligonucleotide
gcattctaatagcagc

SEQ ID NO: 14 - DNA, 18 bp, Artificial
  oligonucleotide
ttgggtgggtgggtgggt

SEQ ID NO: 15 - protein, 12 aa, Artificial
  Peptide
KALVILPETGLE

SEQ ID NO: 16 - protein, 38 aa, Artificial
  Peptide
  MISC_FEATURE (2)..(2): X = Aib
HXEGTFTSDVSSYLEGQAAKEFIAWLVKGRGLPETGLE

SEQ ID NO: 17 - protein, 8 aa, Artificial
  Peptide
KALVINGL

SEQ ID NO: 18 - protein, 34 aa, Artificial
  Peptide
  MISC_FEATURE (2)..(2): X = Aib
HXEGTFTSDVSSYLEGQAAKEFIAWLVKGRGNGL

SEQ ID NO: 19 - protein, 150 aa, Artificial
  Sortase 7M
MQAKPQIPKDKSKVAGYIEIPDADIKEPVYPGPATREQLNRGVSFAKENQSLDDQNISIAGHTFIDRPNYQFTNLKAAKKGSMVYFKVGNETRKYKMTSIRNVKPTAVEVLDEQKGKDKQLTLITCDDYNEETGVWETRKIFVATEVKLE

SEQ ID NO: 20 - protein, 537 aa, Artificial
  His-tag-Ubiquitin-OaAEP1b C247A
MGMAHHHHHHMQIFVKTLTGKTITLEVEPSDTIENVKAKIQDKEGIPPDQQRLIFAGKQLEDGRTLSDYNIQKESTLHLVLRLRGGARDGDYLHLPSEVSRFFRPQETNDDHGEDSVGTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWAYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDADLLHLWEKFRKAPEGSARKEEAQTQIFKAMSHRVHIDSSIKLIGKLLFGIEKCTEILNAVRPAGQPLVDDWACLRSLVGTFETHCGSLSEYGMRHTRTIANICNAGISEEQMAEAASQACASIP